SEQUENCE LISTING (75 sequences)

SEQ ID NO: 1 - DNA, 590 bp, Felis catus
  source 1..590: mol_type = genomic DNA
atggagtttgtgctgggctgggttctcctggttgctcttttaaaaggtgtccagtgtgacgtgcagctggtggagtctgggggagacctggtgaagcctggggggtccctgagactcacctgtgtagcctctggattcaccttcagtagttccagcatgaactgggtccgccaggctccagggaaggggctgcagtgggtcgcatatatttatgatgatggaagtagcacatactacgcagactccgtgaagggccgattcaccatctccagagacaacgccaagaacacgctgtatctgcagatgaacaacctgaagaccgaggacacggccacatattactgtgcaagagacacggtgaggggacctcactgggagcccagacacaaacctccctgcaggaggggatcagcaccaccagggggcgcacactatgcacaactctggtttgtgttcccaggagcaggtgcagatggaggttacaggcaggtttcctgtcagggtctggggcttcctctccacacagcagtttccccagggagcctctctggacacaggattctgtactttcctgttcattccttgactta

SEQ ID NO: 2 - DNA, 591 bp, Felis catus
  source 1..591: mol_type = genomic DNA
atggagtttgtgctgggctgggttttcctggttgctcttttaaaaggtgtccagtgtgacgtgcagctggtggagtctgggggagacctggtgaagcctggggggtccctgagactcacctgtgtggcctctggattcaccttcagtagctatggaatgagctgggtccgccaggctccagggaaggggctgcagtgggtcgcatatattagatatgatggaagtagcacatactacgcagactccgtgaagggccgattcaccatctccagagacaacgccaagaacacgctgtatctgcagatgaacagcctgaagaccgaggacacggccacatattactgtgcaagagacacgctgaggggacctcactgggagcccacacacaaacctccctgcaggaggggatcagcaccaccagggggcgcacactatgcacaattctggtttgtgttcccaggagcaggtgcagatggaggttacaggcaggtttcctgtcagggtctggggcttcctctccacacagcagtttctccagggagcctctctggtcacaggattctgtgtttatctattaactctctgaattag

SEQ ID NO: 3 - DNA, 591 bp, Felis catus
  source 1..591: mol_type = genomic DNA
atggagtttgtgctgggctgggttttcctggttgctcttttaaaaggtgtccagtgtgacgtgcagctggtggagtctgggggagacctggtgaagcctggggggtccctgagactcacctgtgtggcctctggattcaccttcagtagctactacatgaactgggtccgccaggctccagggaaggggctgcagtgggtctcatggattaatactgatggaagtagcacaagctacgcagactccgtgaagggccgattcaccatctccagagacaacgccaagaacacgctgtatctgcagatgaacagcctcaagaccgaggacacggccacatattactgtgcgagagacacagtgaggggacctcactgggagcccagacacaaacctccctgcaggaggggatcagcaccaccagggggcgcacactatgcacaattctcctttgtgttcccaggcgcaggtgcagatggaggttacaggcaggtttcctgtcagggtctggggcttcctctccacacagcagtttccccagggagcctccctggttatatgattctgtgtttacctattaactctctgaattag

SEQ ID NO: 4 - DNA, 644 bp, Felis catus
  source 1..644: mol_type = genomic DNA
atgcagatgctgtggtccctcctctgcctgctggcagctcccctgggtgtcctatctcaacttacacttcgggagtccggcccaggactggtgaagccttcacaatccctctctctcacctgcgttgtctccggaggctctgttaccagcagttactactggaactggatccgccagcgccctgggagagggttggagtggctggggtactggtcaggtagcaccagctacaacccggctttccagggccgcatctccatcactgctgacacagcccagaaccagttctccctgcagctgagctccatgaccaccgaggacacggccgtgtattactgtgcaagaagcacagtgagggaaagtcagtgtgagctcagtcacaaaccttggtgcagggacctggaggggctgggctgcaggggcgctcaggatccacaagagggcacacaggacctaccaggggaactagggcatcagggggtgcttagggccccttaccacagggaccagcccagaaacaggggcagagcaggagtgaggtccccactgtcagtatctggagctttctcttcctggcactctgatcctatggggacctccctttctttcttgcttgcgttcccttttgtttcagtcccagtgtg

SEQ ID NO: 5 - DNA, 19 bp, Felis catus
  source 1..19: mol_type = genomic DNA
gcatagcggaagctggtcc

SEQ ID NO: 6 - DNA, 18 bp, Felis catus
  source 1..18: mol_type = genomic DNA
ggtagtagcgggtgggct

SEQ ID NO: 7 - DNA, 22 bp, Felis catus
  source 1..22: mol_type = genomic DNA
ttactacgatagcgactatgcc

SEQ ID NO: 8 - DNA, 20 bp, Felis catus
  source 1..20: mol_type = genomic DNA
tctataactacgggtggtac

SEQ ID NO: 9 - DNA, 54 bp, Felis catus
  source 1..54: mol_type = genomic DNA
cctatgattacttccagttttggggccagggcaccctggtcaccgtctcctcag

SEQ ID NO: 10 - DNA, 50 bp, Felis catus
  source 1..50: mol_type = genomic DNA
caatacttttggtatctggggccaaggtacccaggtcaccgtctcccaag

SEQ ID NO: 11 - DNA, 48 bp, Felis catus
  source 1..48: mol_type = genomic DNA
actactttgactactggggccaaggagccctggtgacggtgtcctcag

SEQ ID NO: 12 - DNA, 48 bp, Felis catus; identical to SEQ ID NO: 11
  source 1..48: mol_type = genomic DNA

SEQ ID NO: 13 - DNA, 54 bp, Felis catus
  source 1..54: mol_type = genomic DNA
attactacggtatcgatctctggggccatggaaccatagtcacagtgtcctcag

SEQ ID NO: 14 - DNA, 345 bp, Felis catus
  source 1..345: mol_type = genomic DNA
atgaaggcccccgctcagctcctgggcctcctgctgctctggctcccaggagccagctgcgaaatccagatgacccagtctccatcctcgctgtctgcatctccaggagacagagtcaccatcacctgccgggcgagtcagaacgttaacacgtggttagcctggtatcagcagaaaccggggaaagttcctaagcttctgatctatcgtgcatccacgttgcaaactggggtcccctcgcggttcagcggcagtgggtctgggacagatttcaccctcaccatcagcagcctggagcctgaagacgctgccacttactactgtcagcagcataacagcggcatc

SEQ ID NO: 15 - DNA, 347 bp, Felis catus
  source 1..347: mol_type = genomic DNA
atgaaggcccccgctcagctcctgggcctcctgctgctctggctcccaggagccagctgtgaaatccagatgacccagtctccatcctcgctgtctgcatctccaggagacagagtcaccatcacctgccgggcgagtcagaatgttaacacgtggttagcctggtatcagcagaaaccggggaaagttcctaagcttctgatctatcgtgcatccacgttgcaaactggggtcccctcgcggttcagcggcagtgggtctgggacagatttcaccctcaccatcagcagcctggagcctgaagacgctgccacttactactgccagcaaagtagcaatctccctcc

SEQ ID NO: 16 - DNA, 362 bp, Felis catus
  source 1..362: mol_type = genomic DNA
atgaggttccctgctcagctgctggggctgctgatgctctggatcccaggatccagtggggatgtcgtgatgacgcagacccctctgtccctgcccgtcacccctggagagccggcctcaatctcctgcagggccagtcagagcctcctgcacagtaatggaaatacttatctgaattggtacctgcagaagccaggccagtctccacggcgactgatctataaggtttccaaccgggactctggggtcccagacaggttcagtggcagcgggtcagggacagatttcaccctgagaatcagcagggtggaggctgacgacgtcggagtttattactgccagcaaggtacacatgctcctcg

SEQ ID NO: 17 - DNA, 362 bp, Felis catus; identical to SEQ ID NO: 16
  source 1..362: mol_type = genomic DNA

SEQ ID NO: 18 - DNA, 362 bp, Felis catus
  source 1..362: mol_type = genomic DNA
atgaggttccctgctcagctgctggggctgctgatgctctggatcccaggatccagtggggatgtcgtgatgacgcagacccctctgtccctgcccgtcacccctggagagccggcctcaatctcctgcagggccagtcagagcctcctgcacagtaatggaaacacctatttacattggtacctgcagaagccaggccagtctccacggcgactgatctatagggtttccaaccgggactctggggtcccagacaggttcagtggcagcgggtcagggacagatttcaccctgagaatcagcagggtggaggctgacgacgtcggagtttattactgcctgcaaggtacacacagacctcc

SEQ ID NO: 19 - DNA, 362 bp, Felis catus
  source 1..362: mol_type = genomic DNA
atgaggttccctgctcagctcctgggactcatcatgctctggatcccaggatccagtggggatattgtgatgacgcagacccctctgtccctgtccgtcacccctggagagccagcctcaatctcctgcagggccagtcagagcctcctgcacagtgatggaaatacttatctgaattggtacctgcagaagccaggccagtctccacggcgcttgatctatcttgtttccaaccgggactctggggtcccagacaggttcagtggcagcgggtcagggacagatttcaccctgagaatcagcagggtggaggctgacgacgtcggtgtttattactgcggtcaagctttacagtatcctcc

SEQ ID NO: 20 - DNA, 362 bp, Felis catus
  source 1..362: mol_type = genomic DNA
atgaggttccctgctcagctcctggggctgctagtgctttggttccctggatccggtgcggatgtcgtgatgacacagacccctctgtccctgcctgtcacccctggagagccggcctcaatctcctgcagggccagtcagagcctcctgcacagtgatggaaatacttatctgaattggtacctgcagaagccaggccagtctccacggcgactgatctataaggtttccaaccgggactctggggtcccagacaggttcagtggcagcgggtcagggacagatttcaccctgagaatcagcagggtagaggctgacgacgtcggagtttattactgcctgcaaggtacaaagtatcctac

SEQ ID NO: 21 - DNA, 362 bp, Felis catus
  source 1..362: mol_type = genomic DNA
atgaggttccctgctcagctcctggggctgctagtgctttggttccctggatccagtggggatgtcatgatgacccagacccttctgtccctgcccgtcgcccctggacagccgggctcaatctcctgcagggccagtcagagcctcctgcccagcaatggatactcctatttaagttggtacctgcagaagacaggccagtctccacagcgcccgatctatcaggtttccaaccgggcctctggggtcccagacaggttcagtggcagcgggtcagggacagatttcacactcaaaatcaacagagtggaggctgaggatgtgggagtttattgctgcttgcaagatatacaacttcctct

SEQ ID NO: 22 - DNA, 347 bp, Felis catus
  source 1..347: mol_type = genomic DNA
atggtgacccagaggcaggtcctcctatccttgttgctgtgggtctcaggtgcctgtggggcgatcacgatgacgcagtctccaggctccctggctgggtctccaggtcagcaggtcaccatgaactgcagggccagtcagagtgttagcagctacttagcctggtaccagcagaaaccagggcagcatcctaagctgctcatctactcagcttccacccgggcatctggagtccccgaccgattcagtggcagtgggtccgggacggatttcaccctcaccatcagcaacctccaggctgaagacgtggcgagttactactgtcagcagtattacagctctcctcc

SEQ ID NO: 23 - DNA, 347 bp, Felis catus
  source 1..347: mol_type = genomic DNA
atggtgtctccatcacagcttcttgggcttctgctcctctgggttccagcctccagtggtgaggttgtgctgacccagtcctcagccttcctgtccaggactctaaaagaaaaagccaccatcacctgccgagccaatcagggcatcagcaccatcttgcactggtatcagcagaaaccaaatcaggctccgaagctccttgtgaagtatgcttcccagtccgtctcgggagcgccgtcgcggttcagcggcagtgggtctgggacagatttcaccctcaccatcagcagcccggagcctgaagacgctgccacttactactgccagcaaagtaacaatctccctcg

SEQ ID NO: 24 - DNA, 347 bp, Felis catus
  source 1..347: mol_type = genomic DNA
atgggggtcctgacccagctcctctgccttctgctggcctgcctcccagctgccggcgggaccactgagttgacccagtctcccacccatctctctgtgtccctgacagacagcgtgtccgtcatctgcagggccagtgagagcattagtgatcacttaagctggtatcagcagaaaccaggccagcctcccaagcttatcatctatgatgccgataacctagagtctggcgtctcagaccgcttctctgggattcagtctggcacagaattcatcctcaaaatcagcacagtcgaggctgatgacgccgccgcttattactgccagcagggttatgcgcttcctcc

SEQ ID NO: 25 - DNA, 347 bp, Felis catus
  source 1..347: mol_type = genomic DNA
atgggggtcctgacccagctcctctgccttctgctggcctgcctcccagctgccagcgggaccgctgagttgacccagtctcccacccatctctctgtgtccctgacagacagcgtgtccctcatctgcagggccaatgagagcgttagtgattacttaagctggtatcagcagaaaccaggccagcctcccaagcttatcatctatgatgccgataatctagagtctggcgtctcagaccgcttctctgggattcagtctgacacagaattcatcctcaaaatcagcacagtcgaggctgatgacgccgccatttattactgccagcaggattatgcgcttcctcc

SEQ ID NO: 26 - DNA, 38 bp, Felis catus
  source 1..38: mol_type = genomic DNA
gtggacgtttggccaaggaaccaagctggaagtcaaac

SEQ ID NO: 27 - DNA, 39 bp, Felis catus
  source 1..39: mol_type = genomic DNA
tgtacaatttcggccaggggacgaagctggagataaaac

SEQ ID NO: 28 - DNA, 38 bp, Felis catus
  source 1..38: mol_type = genomic DNA
gttcactttcggccaggggaccaaactggagatgaaac

SEQ ID NO: 29 - DNA, 38 bp, Felis catus
  source 1..38: mol_type = genomic DNA
gctcactttcggcccaggtaccaagctggagatcaaac

SEQ ID NO: 30 - DNA, 38 bp, Felis catus
  source 1..38: mol_type = genomic DNA
gaccacctttggccaagggacacatctggagattaaac

SEQ ID NO: 31 - DNA, 353 bp, Felis catus
  source 1..353: mol_type = genomic DNA
atggcttggtctccggtccttctcaccctcctcgctcactgcacagggtcctgggcccagtctgtactggctcagccatcctcggtgtcaggctccttgggccagagggtcaccatctcctgctctggaagcagttccaacatcggtagcaattatgtgagctggtaccaacaactcccaggcacaacccccaaaaccataatctattgggataatagcagaccctcgggggtctctgaacgattctctggctccaagtctggcagcacaggcaccctgaccatcactgggctccaggctgaggacgaggctgattattactgctcagcatgggatggtagtctgagagctca

SEQ ID NO: 32 - DNA, 353 bp, Felis catus
  source 1..353: mol_type = genomic DNA
atggcctggtctcctctcctcctcatcctcctcgctcactgcacagtgtcctgggcccagtctaggctgactcagccgccctcagtgtctggttctctgggccagagggtcaccatctcctgcgctggaagcagctctaatattggtggttatggtgtgaactggcaccaacaattcccaggaatggcccccaaaaccatcatctatggtaatagcaatcgaccctctggggtcccagatcgattctccggctccaagtctggcaacacaggcaccctgaccatcactgggctccaggctgaggacgaggctgattattactgctcatcgtgggacagcagtagcagtgctcg

SEQ ID NO: 33 - DNA, 353 bp, Felis catus; identical to SEQ ID NO: 32
  source 1..353: mol_type = genomic DNA

SEQ ID NO: 34 - DNA, 353 bp, Felis catus
  source 1..353: mol_type = genomic DNA
atggcctggtcccctctcctcctcaccctcctcattcactgcacagggtcctgggcccagtctgtgttgagtcagccaccctcagtgtctggggccctgggccagacggtcaccatcccctgcgctggaggtgccaacaacatcggtattgctggtgggaactggtaccaacagcttccaggaaaggcccctaaactcctcatctatgatagtagcgatcgaccctcaggggtccctgaacgattctctggctccaagtctggcaacacaggctccttgaccatcactgggctccaggctgaggacgaggctgattattactgccagtctcttgactttactcaaggtgctga

SEQ ID NO: 35 - DNA, 356 bp, Felis catus
  source 1..356: mol_type = genomic DNA
atggcctggacccctcttctcctcaccctacttgctcactgtacagggtcctgggcccagtctgtgctgactcagccaccatcagtgtctgggaccctaggccagaggatcaccatctcctgcaccggaagcagctccaacatcgggggtggtaatgctgtgagctggtaccaacaagtcccaggaatgggccccaaaaccgtcatctattggaataacagcaaaccctcgggggtcccagatagattctccggctcaaagtctggcagttcaggcaccctgaccatcactgggctgcaggctgaggacgaggctgattattactgctcagcgtgggatgatagtctcagtgctca

SEQ ID NO: 36 - DNA, 356 bp, Felis catus
  source 1..356: mol_type = genomic DNA
atggcctggtctcctttccttctcactctcctcgctcactgcacagggtcctgggcccagtctgtgctgactcagccgccctcagtgtcggggtccctgggccagagggtcaccatctcctgcactggaagcagctccaacatcgggggtggtaattatgtgagctggtaccaacaagtcccaggaacggcccccaggctcctgatttatgagaataacaaacgaccctccggggtccccgatcgattctctggctccaagtctggcagctcaggctccctgaccatcactgggctgcaggctgacgacgaggctgattattattgtgcatcatgggacaatagtctcagtgctca

SEQ ID NO: 37 - DNA, 365 bp, Felis catus
  source 1..365: mol_type = genomic DNA
atggcctggttccctcttctcctgacccttctcatccactgcacagggtcctgggcccagtctgtgctgactcagccgccctcagtgtctggggccctggggcagacggtcaccatctcctgcgctggaagtaggagcaacatcggtattgctggtgtgaactggtaccaacagcttccaggaaaggcccctaaactcctctctatgctaatagacagaaacaaccctcatgggttccctgaacgaatccgcgaatctcctggctccaagtctggcaacacaggctccttgaccatcactgggctccaggctgaggacgaggctgattattactgctcagcatgggatgctattctgaaagctca

SEQ ID NO: 38 - DNA, 356 bp, Felis catus
  source 1..356: mol_type = genomic DNA
atggcctggtcctttctcctcctcaccctcctcgctcactgcacagggtcctgggctcagtctgtgctgactcagccgccctcagtgtctggggccctgggccagagggtcaccatctcctgcactggaagcagctccaacatcgggcgtggtaattatgtgagctggtaccaacaactctcaggaacagctcccaaactcctcatctatggtaatagcaatcgaccctcgggggtcccagatcgattttctggctccaagtctggcagcacaggctccttgaccatcactgggctgcaggctgaggacgaggctgattattactgtgcagcgtgggacagcagtctcaatgctca

SEQ ID NO: 39 - DNA, 353 bp, Felis catus
  source 1..353: mol_type = genomic DNA
atgtcctggtctcctgtccttctcgccctcttcactcactgcacagggtcctgggcccagtctgtactgactcagccaccctcggtgtcaggctccttgggccagagggtcaccatctcctgcactggaagcagctcccacatcagtaacaattttgtgaactggtaccaacaactcccaggcacaacccccaaaaccataatcctttgggatgatagcagaccctcgggggtctctgaacgattctctggctccaagtctggcagcacaggcaccctgaccatcactgggctccaggctgaggacgaggctgattattactgctcagcatgggatgatagtctgagagctca

SEQ ID NO: 40 - DNA, 353 bp, Felis catus
  source 1..353: mol_type = genomic DNA
atggcctggttccctcttctcctcaccctcctcatctactgcacagggtcctgggcccagtctgagctgactcagccgccctcagtgtctgggggcctgggccagacggtcaccatctcctgcgctggaagtaggagcaacattggtattgctggtgtgaactggtaccaacagcatccaggaaaggcccctaaactcctcatctatggtagtagcaatcgaccctcaggggtccctgacagattttctggctccaagtctggcaacacaggctccttgaccatcactgggctccaggccgaggacgaggctgattattattgcctgtctgttgacgttacgcgaggtgctga

SEQ ID NO: 41 - DNA, 353 bp, Felis catus
  source 1..353: mol_type = genomic DNA
atggcctggttccctcttctcctcaccctcctcatctactgcacaggttcctgggcccagtctgagctgactcaaccgccctcagtgtctgggggcctgggccagacggtcaccatctcctgcgctggaagtgccaacaacatcggtagaattggtgtgaactggtaccaacagtttccaggaaaagcccctaaactcctcatctccgcttatagcaatcaaccctcacgggtttctggtcgattttctggctccacgtctggcaacacaggctccttgaccatcactgggctccaggccgaggacgaggctgattattactgcacgtctgctgaccctattcaaagtgctca

SEQ ID NO: 42 - DNA, 354 bp, Felis catus
  source 1..354: mol_type = genomic DNA
atggcctgggctctggtcctcctcagtctcctcactcaggacacagggtcctgggcccagtctgccctgaatcagcctccctcactgtccggggatctgggacgcacagtcaccatctcctgtgctggcagcagcaatgacattgggagatatagtgacgtctcctggtaccaacagctcgaaggcacatcccccaaactcctgattcataatgtaaattcccggccttcagggatccctgatcgcttctctggctccaagtctggcaacacggcctccttgaccatctctgggctccaggctgaagatgaggctgattattactgttgctcatatgctagtagtaatactctc

SEQ ID NO: 43 - DNA, 345 bp, Felis catus
  source 1..345: mol_type = genomic DNA
atggcctggacccctcttctcctcagtatcctggctcactgcacaggttccatggcctcctacgtgctgacccagcccccgtcggtgtcagtgaacctgggacagacagccagaatcacctgtgggggaaacaacattggaagtaaacatgcttactggtaccagcagaagccaggccaggcccccatgctggtcatttactatagcagcaaccggccctcagggatccctgaccgattctctggcaccaactcggggaacacggccaccctgaccatcagcggggcccaggctgaggacgaggctgactattactgtcaggtgtgggataacagtggtaatgct

SEQ ID NO: 44 - DNA, 345 bp, Felis catus
  source 1..345: mol_type = genomic DNA
atggcctggacccctctcctcctcagtgtcctggcatactacacaggctccgtgacctcaaacagggtgactcagcccccttccatttcagtggccctgggagagatggcaaggatcacctgtgagggaaacaacatcggaaatacatatgtttcctggtaccagcagaagccgaaccaggtgcccctgatgattatttatcaggatagcaaccggccttcagggatccctgaccgattctctggctctaactctgggaacacggccaccctgactgtcagcggggcccgggctgaggatgaggctgactattactgtctgtctgctcacagcagtagtaacgtt

SEQ ID NO: 45 - DNA, 345 bp, Felis catus
  source 1..345: mol_type = genomic DNA
atggcctggacccctctcctcctcggcctcctcgctcactgcacaggttctgtggcttcctatgagctgactcagcccccatcagtgtcagtgaacctgggacagacggccaggatcacatgtggaggaaacaacattggaaataaagatgcttactggtaccagcagaagtcaggccaggcccccatgctgattatctatgaggacagcaaacggccctcagggatccctgaccgattctctggcaccaactcagggaacatggccaccttgaccatcagcggggcccgggccgaagatgaggctgactattactgccaggtgtgggacagcagtagtgatgct

SEQ ID NO: 46 - DNA, 345 bp, Felis catus
  source 1..345: mol_type = genomic DNA
atggcctggacccctctcatcctcagcctcctcacttactgcacaggttccatagcctcctatgtgctgactcagcccccctcagtgtcggtgagcctgggacagacggccaggaccacctgtggaggaaacaacattgaaagcaaaagtgttcactggtaccagcagaagtcaggccaaacccctgtgctgattatctatagtgatagcaaccggccctcaggaatccccgaccgattctcaggcaccaactcggggaacacggccaccctgaccatcagcggggcccgggccgaggacgaggctgactattactgtcaggtgtgggacagtagtagtgatgct

SEQ ID NO: 47 - DNA, 345 bp, Felis catus
  source 1..345: mol_type = genomic DNA
atggcctggacccctctcctgctccccctccttactctttgcacaggattcgtggcctccagtgaggtgactcagccgccctcagtgtcagtggccctgggacagacggctagaatcacctgctctggagatatgatggagaaaaaatataccaattggcaccagcagaagccaggtcaagcccccatacagatcatttataaggatagtgagcggccctcagggatccctgaccgattctctagctccagttcagggaaaacagtcaccctgaccatcagcggggcccgggccgaagacgaggctgactactactgtcagtcttatgacatcagtagtaatgct

SEQ ID NO: 48 - DNA, 345 bp, Felis catus
  source 1..345: mol_type = genomic DNA
atggcctggacccctctcctcctcggcctcctcgctcactgcacaggctccgtggcctcctatgtgctgactcagcccccatcagtggcagtgaacctgggacagacggccaggatcacatgtggaggaaacaacattggaagtagttatgcttactggtaccagcagaagtcaggccaggcccctgtgctgattatctataaggatagcaaccggccctcagggatccctgaccgattctcaggcaccaactcggggaacacggccaccctgaccatcagcggggcccgggccgaggacgaggctgactattactgtcagtcatatgacagcaactatgatcct

SEQ ID NO: 49 - DNA, 345 bp, Felis catus
  source 1..345: mol_type = genomic DNA
atggcctggactcctctcctgctccccctccttattctctgcacaggttctgtgacagcttctgaactgactcagccacctgcggtgtctgtggccttgggacagacggccacgattacatgcgagggagacagcttcgaaagcagtatgattaactggtatcagcagaaatcaggccaagcccccgtgctggtcatttatgaggatagtgagcagcccacagggattcctgacagattctctggctccaactcgggggatgcagccactctgaccatcaccggggcccaggctgaggacgaggctgactattactgtcagtcctatgataacagtggtgatgct

SEQ ID NO: 50 - DNA, 345 bp, Felis catus
  source 1..345: mol_type = genomic DNA
atggcctggacccctctcctccttggcctcctcgctcactgcacaggctccgtggcctcctatgtgctgactcagcccccatcagtggcagtgaacctgggacagacagccaggatcacatgtggaggaaacaacattggaagtagttatgcttactggtaccagcagaagtcaggccaggcccctgtgctgattatctatgaggacagcaaacggccctcagggatccctgaccgattctcaggcaccaactcggggaacacggccaccctgaccatcagcggggcccgggccgaggacgaggctgactattactgtcaggtgtgggataacagtggtaatgct

SEQ ID NO: 51 - DNA, 345 bp, Felis catus
  source 1..345: mol_type = genomic DNA
atggcctggacccctctcctcctcggcctcctcgcttactgcacaggctccatggcctcctacgtgctgactcagcccccgtcagtgtcaatgaacctgggaaagacggccaggatcacgtgtggaggaaacaacattggaagtaaatatgcttactggtaccagcagaagccaggccgggccccgatgatgattatctatgatgatagcaaaaggtcctcaaggacccctgaccgattctcaggcaccaactcggggaacacggccaccctgaccatcagcggggcccaggccgaggacgaggccaactattactgtcaggtgtggggtggcaatagtgatcct

SEQ ID NO: 52 - DNA, 371 bp, Felis catus
  source 1..371: mol_type = genomic DNA
atggactgggctcccttctacctcctgcccttcattttctctacaggtttctgtgctctgcctgtgctgacccaggctccatctgcatctgcctcacttggagcctcagtcaagctcacctgcaccctgagcagtgaacacagcaattactttgtttggtggtatcaacagagaccagagaaggcccctcggtatttgatgaaggttaacagtgatggaagccacatcaaaggagacggaatccccagtcgcttctcaggctcaagctctggggctgatcgctacttaaccatctccaacatccagcctgaggatgaggctgactattactgtggtgagaagcatacaattgatggtcaaaccggttaagc

SEQ ID NO: 53 - DNA, 366 bp, Felis catus
  source 1..366: mol_type = genomic DNA
atggcctggattcctcccctcctagtgctcctctgtcactgcacatgttccatctcccaatttgtggtgacccagccaccttccctctctgcatctctgggaacaacagccagactcacctgcaccctcaacagtgaaaggactgatatttaccccatattctggtaccagcaaaagccagggagccttcctcgttacctccttacctatgtaacagactcaaataagcatcaaggctctggggtccccagccgcttctctggatccaaagatacctctgccaatgcagggattttgctcatttccgggattcaatcagaggatgaggcagactattactgtcagtcatttgatgttggtgacgga

SEQ ID NO: 54 - DNA, 369 bp, Felis catus
  source 1..369: mol_type = genomic DNA
atggcctggatccccatcctcctcgtgttcctctgtcactgcacaggttccctgtcccagcctgtcgtgactcagccagcctccctctctgcatctctgggagccacagccagactcacctgcaccctgagcagggacatcaatgctggaggctactacatatactggtaccaacagaagccagggagccctccccggtatctcctgtactactactcagattcaaataagcaccagggccccggggtccccagccgcttctccgggtccaaagatgcctcggccaatgcagggcttctgctcatctctgggctgcagcctgaggacgaggctgactattactgtgcaacttggcacagtagtgctggt

SEQ ID NO: 55 - DNA, 382 bp, Felis catus
  source 1..382: mol_type = genomic DNA
atggcctggacccatgctctcctcgtgctcttctgccactgcacaggttccctgtcccagcctgtcgtgactcagccaccctccctctctgcatctctgggagcaacagccagactcacctgcaccctcagcagggaagtgagcgttggtagtaaaagcatatactggtaccaacagaagccagggagccctcctcggtatttcctgtactactactcagactccagcaatgagctgggacccggggtccccagtcgagtgtctgggtccaaagatgcctcggccaatgcagggcttctgctcatctctgggctgcagcctgaggacgaggctgactattactgtgctatagcacatggcagtggaagcagctttggttact

SEQ ID NO: 56 - DNA, 382 bp, Felis catus
  source 1..382: mol_type = genomic DNA
atggcttggactcttctagtcctcatgcttgtgtctcaatggacaggttccctgtcccagcctgtgctgacccagccgtcctccctgtctgcatctcctggaacaacagccagactcacctgcaccctgagcagcgggttcagtgttggaggctactacataaattggttccagcagaagccagggagccctccccggtatctcctgtactactactcagattcaaataagcaccagggccctggggtccccagccgcttctccgggtccaaagatgcctcggccaatgcagggcttctgctcatctctgggccgcagcctgaggacgaggctgactattactgtgctatagcacatggcagtggaagcagctaccgttact

SEQ ID NO: 57 - DNA, 382 bp, Felis catus
  source 1..382: mol_type = genomic DNA
atggcctggatccccgtcctcctcgtgctcctctgtcactgcgcaggttccctgtcccagcctgtcgtgactcagccaccctccctctctgcatctctgggagcaacagccagactcacctgcaccctcagcagggaagtgagcgttggtagtaaaagcatatactggtaccaacagaagccagggagccctcctcggtatttcctgtactactactcagactccagcaatgagctgggacccggggtccccagtcgagtgtctgggtccaaagatgcctcggccaatgcagggcttctgctcatctctgggctgcagcctgaggacgaggctgactattactgtgctatagcacatggcagtggaagcagctaccgttact

SEQ ID NO: 58 - DNA, 370 bp, Felis catus
  source 1..370: mol_type = genomic DNA
atggcctggactcctgtcctcctcatgctcctctcccattgtgcaggttccctgtcccagcctgtgctgacccagccgtcctccctgtctgcatctccgggaacaacagccagactcacctgcaccctgagcagcggcttcaatgttggaggctactacataagttggttccagcagaagccagggagccctccccggtatctcctgtactactactcagactcagataagcaccagggccccggggtccccagccgcttctccgggtccaaagatgcctcggccaatgcagggcttctgctcatctctgggccgcagcctgaggacgaggctgactattactgtgctacagatcatggcagtggttact

SEQ ID NO: 59 - DNA, 382 bp, Felis catus
  source 1..382: mol_type = genomic DNA
atggcttggcctcttctcgtcctcatactcctgtctcactgcacaggttccctgtcccagcctgtgctgacccagccgccctccctgtctgcatctccgggaacaacagccagactcacctgcaccctgagcagcggcttcaatgttggaggctactacataagttggttccagcagaagccagggagccctccccggtatctcctgtactactactcagactcagataagcaccagggccccggggtccccagccgcttctccgggtccaaagatgcctcggccaatgcagggcttctgctcatctctgggccgcagcctgaggacgaggctgactattactgtgctacaggttatggcagtgggagcagctaccgttact

SEQ ID NO: 60 - DNA, 369 bp, Felis catus
  source 1..369: mol_type = genomic DNA
atggcctggatctccgtcctcctcgtgctcctctgtcactgcgcaggttccctgtctcagcctgtcgtgactcagccagcctccctctctgcatctctgggagcaacagccagactcacctgcacgctgagcagggacatcaatgttggaggctactacatatactggtaccaacagaatccagggagccctccccggtatctcctgtactactactcagactccagtacacagttgggacctggggtccccagccgcttctccggatccaaagatgcctcggccaatgcagggcttctgctcatctctgggctgcagcctgaggacgaggctgactattactgtgcaatcgggcacagtagtgctggt

SEQ ID NO: 61 - DNA, 369 bp, Felis catus
  source 1..369: mol_type = genomic DNA
atggcctggatccccatcctcctcgtgctcctctgtcactgcacaggttccctgtcccagcctgtcttgactcagccagcctccctctctgcatctctgggagcaacagccagactcacctgcaccctgagcagggacatcaacgttggaagctataacatatactggtaccaacagaagccagggagccctccccggtatctcctgtactactactcagactcagataagcaccagggccctggcgtccccagccgcttctctgggtccaaagatgcctcggccaatgcagggcttctgctcatctctgggctgcagcctgaggacgaggctgactattactgtgcaatctggcacagtagtgctggt

SEQ ID NO: 62 - DNA, 374 bp, Felis catus
  source 1..374: mol_type = genomic DNA
atggcttggaccccttttttccttgtgttcctggctcactgcacaggttccctgtctcagccggtgctgacccagccaccctccctctctgcatctctgggaacttctgtgagacttacctgtaccctgagcagtggcttcagagttggtgatttctggataaactggtaccagcagaatccagggaaccctccccggtatctcctgtactaccactcagactcagataaacaccagggctccggggtccccagccgcttctctggatccagtgatgcctcggccaatgcagggcttctgctcatctctgggctgcagcctgaggatgaggctgactattactgtagcacatggcatggcaactctaagtctta

SEQ ID NO: 63 - DNA, 351 bp, Felis catus
  source 1..351: mol_type = genomic DNA
atggcctgggctcttctcctgttcacacttctgtctcactgcacaggggccacttcccaggaagtagtgactcaggaaacttcactctcaacaactcctggaggaacagtcacactcacctgtggctccagtactggggctgtcaccaccagtaattatgccagctgggtccaacagaagccctaccagagattccagggtctgataggtgggaccagctaccggaacccaggggtccctgcccgattctctggctccctggttggacagaaggccgtcctcaccatcacgggggcgcagtcagaggatgaagctgagtattactgtgttctgtggttcagcaaccattac

SEQ ID NO: 64 - DNA, 38 bp, Felis catus
  source 1..38: mol_type = genomic DNA
ttgggtgttcggcggaggtacccatctgagcgtcctag

SEQ ID NO: 65 - DNA, 38 bp, Felis catus
  source 1..38: mol_type = genomic DNA
tcatattttcggtggagggacccatctgactgtcctcg

SEQ ID NO: 66 - DNA, 38 bp, Felis catus
  source 1..38: mol_type = genomic DNA
ttatgttttcggcggagggaccaaggtgaccgtcctcg

SEQ ID NO: 67 - DNA, 38 bp, Felis catus
  source 1..38: mol_type = genomic DNA
tcctattttcggcggagggacccgtctgaccgtcctcg

SEQ ID NO: 68 - DNA, 38 bp, Felis catus
  source 1..38: mol_type = genomic DNA
ttttgtttttggcagagggacctggctgacggtcctag

SEQ ID NO: 69 - DNA, 38 bp, Felis catus
  source 1..38: mol_type = genomic DNA
tgctttgttcggcggagggacccatctgaccgtcctcg

SEQ ID NO: 70 - DNA, 38 bp, Felis catus
  source 1..38: mol_type = genomic DNA
ttgggtgtttggcgatggaacccagctgactgtattag

SEQ ID NO: 71 - DNA, 38 bp, Felis catus
  source 1..38: mol_type = genomic DNA
tattgtgttcggcggagggacccatctgaccgtcctcg

SEQ ID NO: 72 - DNA, 38 bp, Felis catus; identical to SEQ ID NO: 70
  source 1..38: mol_type = genomic DNA

SEQ ID NO: 73 - DNA, 38 bp, Felis catus; identical to SEQ ID NO: 71
  source 1..38: mol_type = genomic DNA

SEQ ID NO: 74 - DNA, 100 bp, Mus musculus
  source 1..100: mol_type = genomic DNA
atttctgtacctgatctatgtcaatatctgtaccatggctctagcagagatgaaatatgagacagtctgatgtcatgtggccatgcctggtccagacttg

SEQ ID NO: 75 - DNA, 100 bp, Mus musculus
  source 1..100: mol_type = genomic DNA
gtcaatcagcagaaatccatcatacatgagacaaagttataatcaagaaatgttgcccataggaaacagaggatatctctagcactcagagactgagcac

(19) United States
(12) Patent Application Publication
(10) Pub. No.: US 2024/0423175 A1
(43) Pub. Date: Dec. 26, 2024

(54) TRANSGENIC MAMMALS AND METHODS OF USE THEREOF

(71) Applicant: TRIANNI, INC., Wilmington, DE (US)

(72) Inventors: Peter BURROWS, Birmingham, AL (US); Werner MUELLER, Köln (DE); Matthias WABL, San Francisco, CA (US)

(21) Appl. No.: 18/707,725

(22) PCT Filed: Nov. 9, 2022
(86) PCT No.: PCT/US2022/079533

§ 371 Date: May 6, 2024

Related U.S. Application Data
(60) Provisional application No. 63/263,855, filed on Nov. 10, 2021.

(51) Int. Cl. A01K 67/0275; C07K 16/46; C12N 5/0781

(52) U.S. Cl. CPC A01K 67/0275; C07K 16/462; C12N 5/0635; A01K 2217/05; A01K 2227/105; A01K 2267/01; C07K 2317/24; C12N 2510/04

(57) ABSTRACT

Described herein are transgenic mammals that express immunoglobulins with feline variable domains, including transgenic rodents that express immunoglobulins with feline variable domains for the development of therapeutic feline antibodies.

### FIELD OF THE INVENTION

This invention relates to production of immunoglobulin molecules, including methods for generating transgenic mammals capable of producing antigen-specific antibody-secreting cells for the generation of feline monoclonal antibodies.

### BACKGROUND OF THE INVENTION

In the following discussion certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Antibodies have emerged as important biological pharmaceuticals because they (i) exhibit exquisite binding properties that can target antigens of diverse molecular forms, (ii) are physiological molecules with desirable pharmacokinetics that make them well tolerated in treated humans and animals, and (iii) are associated with powerful immunological properties that naturally ward off infectious agents. Furthermore, established technologies exist for the rapid isolation of antibodies from laboratory animals, which can readily mount a specific antibody response against virtually any foreign substance not present natively in the body.

In their most elemental form, antibodies include two identical heavy (H) chains that are each paired with an identical light (L) chain. The N-termini of both H and L chains include a variable domain (V_{H} and V_{L}, respectively) that together provide the paired H-L chains with a unique antigen-binding specificity.

The exons that encode the antibody V_{H} and V_{L} domains do not exist in the germ-line DNA. Instead, each V_{H} exon is generated by recombination of randomly selected V_{H}, D_{H}, and J_{H} gene segments present in the immunoglobulin H chain locus; likewise, individual V_{L} exons are produced by the chromosomal rearrangements of randomly selected V_{L} and J_{L} gene segments in a light chain locus.

In mammals, the genome typically contains two alleles that can express the H chain, two alleles that can express the kappa (κ) L chain, and two alleles that can express the lambda (λ) L chain (one allele from each parent). There are multiple V_{H}, D_{H}, and J_{H} gene segments at the immunoglobulin H chain locus as well as multiple V_{L} and J_{L} gene segments at both the immunoglobulin κ (IGK) and immunoglobulin λ (IGL) L chain loci (Collins and Watson (2018) Immunoglobulin Light Chain Gene Rearrangements, Receptor Editing and the Development of a Self-Tolerant Antibody Repertoire. Front. Immunol. 9:2249. (doi: 10.3389/fimmu.2018.02249)).

In the heavy chain locus, exons for the expression of different antibody classes (isotypes) also exist. For example, in feline animals, the encoded isotypes are IgM, IgD, IgG1a, IgG2, IgE, and IgA2.

During B cell development, gene rearrangements occur first on one of the two homologous chromosomes that contain the H chain variable gene segments. In pre-B cells, the resultant V_{H} exon is then spliced at the RNA level to the C_{μ} exons for IgM H chain (μH chain) expression. Most of the μH chain synthesized by pre-B cells is retained in the endoplasmic reticulum (ER) and eventually degraded due to the non-covalent interaction between the partially unfolded C_{H}1 domain of the μH chain and the resident ER chaperone BiP (Haas and Wabl, Nature, 306:387-9, 1983; Bole et al., J Cell Biol. 102:1558, 1986). However, a small fraction of the μH chains associate with a surrogate light chain complex, which includes invariant λ5 and VpreB proteins. This association displaces BiP and allows the μH chain/λ5/VpreB complex, together with an Igα/β signaling molecule heterodimer, to exit the ER as a preB Cell Receptor (preBCR) and traffic through the secretory pathway to the plasma membrane.

Subsequently, V_{L}-J_{L} rearrangements occur on one L chain allele at a time until a functional L chain is produced, after which the L chain polypeptides can associate with the IgM H chain homodimers to form a fully functional antigen-specific B cell receptor (BCR), which is expressed on the surface of the immature B cell.

The immature B cells migrate to secondary lymphoid organs where they differentiate into mature B cells that can respond to cognate antigen and differentiate into antibody-secreting plasmacytes and memory B cells. With the assistance of T cells, the B cells can undergo isotype switching, which changes the antibody isotype from IgM to IgG, IgA or IgE, as well as somatic hypermutation, which can change the amino acid sequence of the V_{H} and V_{L} domains. Although these mutations are introduced randomly into the V_{H} and V_{L} exons, B cells with higher affinity for the immunizing antigen are able to take up more of the antigen, process it and present it to T follicular helper cells and thus are preferentially activated compared to B cells with low or no affinity for the immunizing antigen. As a result, the somatic mutations become enriched in the complementarity determining regions (CDR) 1, 2 and 3, because these are the regions of the V_{H} and V_{L} domains that interact with the antigen.

The genes encoding various mouse immunoglobulins have been extensively characterized. For example, Blankenstein and Krawinkel described the mouse variable heavy chain region in Eur. J. Immunol., 17:1351-1357 (1987). While there is less information about the feline immunoglobulin heavy chain locus, studies of lymphoid malignancies and responses to viruses have included some V_{H} domain sequences [e.g., Rout et al., Vet. Clin. Pat. 45:48 Suppl. 1 (2019) and Lu et al. Scientific Reports 7:12713 (2017)]. Lu et al. also characterized feline IgG1a, IgG2 and IgA sequences. The feline kappa and lambda LC loci have been extensively characterized and are fully annotated in The International ImMunoGeneTics (IMGT) information system.

The generation of transgenic animals—such as mice having varied immunoglobulin loci—has allowed the use of such transgenic animals in various research and development applications, e.g., in drug discovery and basic research into various biological systems. For example, the generation of transgenic mice bearing human immunoglobulin genes is described in International Application Nos. WO 90/10077 and WO 90/04036. WO 90/04036 describes a transgenic mouse with an integrated human immunoglobulin “mini” locus. WO 90/10077 describes a vector containing the immunoglobulin dominant control region for use in generating transgenic animals.

Numerous methods have been developed for modifying the mouse endogenous immunoglobulin variable region gene locus with, e.g., human immunoglobulin sequences, to create partly or fully human antibodies for drug discovery purposes. Examples of such mice include those described in, e.g., U.S. Pat. Nos. 7,145,056; 7,064,244; 7,041,871; 6,673,986; 6,596,541; 6,570,061; 6,162,963; 6,130,364; 6,091,001; 6,023,010; 5,593,598; 5,877,397; 5,874,299; 5,814,318; 5,789,650; 5,661,016; 5,612,205; and 5,591,669.

The use of antibodies that function as drugs is not limited to the prevention or therapy of human disease. Domestic animals such as cats suffer from afflictions similar to those of humans, e.g., cancer, atopic dermatitis and chronic pain. Monoclonal antibodies targeting Nerve Growth Factor (bedinvetmab) are already in veterinary use for treatment of osteoarthritis in cats, but none are yet approved for treatment of cancer or atopic dermatitis. However, before clinical use, the monoclonal antibodies, which were made in mice, had to be felinized, i.e., their amino acid sequence had to be changed from mouse to feline to prevent an adverse immune response in the recipient cats.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Described herein are methods for producing mouse antibodies with feline immunoglobulin variable regions. In one aspect, an antibody with feline variable regions is provided that can be produced in a transgenic mammal or in an in vitro cell culture.

In one aspect, a non-feline mammalian cell or a non-feline mammal is provided that has a genome that includes a heterologous partly feline immunoglobulin locus. In one aspect, the heterologous locus includes coding sequences of the feline immunoglobulin variable region genes and non-coding sequences based on the endogenous immunoglobulin variable region locus of the non-feline mammalian host. In one aspect, the non-feline mammalian cell or mammal is capable of expressing a chimeric B cell receptor (BCR) or antibody that includes feline heavy (H) and light (L) chain variable regions and constant regions that are endogenous to the non-feline mammalian host cell or mammal. In one aspect, the transgenic mammalian host cell or mammal has a genome in which part or all of the endogenous immunoglobulin variable region gene locus has been removed.

To produce chimeric feline monoclonal antibodies in a non-feline mammalian host, the host genome should have at least one locus that expresses chimeric feline immunoglobulin H or L chain. In one aspect, the host genome includes one heavy chain locus and two light chain loci that express chimeric feline immunoglobulin H and L chains, respectively.

In some aspects, the partly feline immunoglobulin locus includes feline V_{H} coding sequences and non-coding sequences present in the endogenous V_{H} gene locus of the non-feline mammalian host. In some aspects, the partly feline immunoglobulin locus includes feline V_{H} coding sequences and non-coding regulatory or scaffold sequences present in the endogenous V_{H} gene locus of the non-feline mammalian host. In one aspect, the partly feline immunoglobulin locus includes feline D_{H} and J_{H} gene segment coding sequences and non-coding sequences present in the endogenous D_{H} and J_{H} gene segments of the non-feline mammalian host cell genome. In one aspect, the partly feline immunoglobulin locus includes feline D_{H} and J_{H} gene segment coding sequences and non-coding regulatory or scaffold sequences present in the endogenous D_{H} and J_{H} gene segments of the non-feline mammalian host cell genome.

In other aspects, the partly feline immunoglobulin locus includes feline V_{L} coding sequences and non-coding sequences present in the endogenous V_{L} gene locus of the non-feline mammalian host. In other aspects, the partly feline immunoglobulin locus includes feline V_{L} coding sequences and non-coding regulatory or scaffold sequences present in the endogenous V_{L} gene locus of the non-feline mammalian host. In one aspect, the heterologous partly feline immunoglobulin locus includes feline V_{L} coding sequences and feline J_{L} gene segment coding sequences and non-coding sequences present in the endogenous J_{L} gene segments of the non-feline mammalian host cell genome. In one aspect, the heterologous partly feline immunoglobulin locus includes feline V_{L} coding sequences and feline J_{L} gene segment coding sequences and non-coding regulatory or scaffold sequences present in the endogenous J_{L} gene segments of the non-feline mammalian host cell genome.

In one aspect, the non-feline mammal is a rodent, for example, a mouse or rat.

In one aspect, a method is provided for generating a non-feline mammalian cell that includes a partly feline immunoglobulin locus. In one aspect, the method includes: a) introducing two or more recombinase targeting sites into the genome of a non-feline mammalian host cell and integrating at least one site upstream and at least one site downstream of a genomic region that includes endogenous immunoglobulin V_{H}, D_{H} and J_{H} genes or endogenous V_{L} and J_{L} genes; and b) introducing into the non-feline mammalian host cell via recombinase-mediated cassette exchange (RMCE) a heterologous partly feline immunoglobulin variable gene locus that includes feline V_{H}, D_{H} and J_{H} gene or feline V_{L} and J_{L} gene coding sequences and non-coding sequences based on the non-coding sequences present in the endogenous immunoglobulin variable region gene locus of the non-feline mammalian host.

In another aspect, the method includes deleting the endogenous immunoglobulin variable region in the genome of the host animal that is flanked by the two heterologous recombinase-targeting sites prior to introducing into the non-feline mammalian host cell via RMCE a heterologous partly feline immunoglobulin variable gene locus.

In one aspect, the heterologous partly feline immunoglobulin locus includes feline V_{H} gene segment coding sequences, feline D_{H} and J_{H} gene segment coding sequences and non-coding regulatory or scaffold sequences upstream of the feline D_{H} gene segments (Pre-D sequences, FIG. 1) based on the sequences present upstream of the endogenous D_{H} gene segments in the genome of the non-feline mammalian host. In one aspect, the upstream scaffold sequences contain non-immunoglobulin genes, such as Adam6a (FIG. 1), which is related to male fertility [Nishimura et al. Developmental Biol. 233(1):204-213 (2011)]. In one aspect, the partly feline immunoglobulin locus is introduced into the host cell using recombinase targeting sites that were previously introduced upstream of the endogenous immunoglobulin V_{H} gene locus and downstream of the endogenous J_{H} gene locus on the same chromosome.

In one aspect, the scaffold sequences include a naturally occurring nucleic acid sequence from another species. In one aspect, the scaffolding sequences can be designed based on a naturally occurring nucleic acid sequence from another species, for example, the scaffolding sequences can include a naturally occurring nucleic acid sequence from another species that has been modified, for example, by one or more nucleic acid substitutions, insertions, deletions or other modifications. In one aspect, the scaffolding sequences can include an artificial sequence. In one aspect, the scaffold sequence includes sequences that are present in the immunoglobulin locus of the feline genome in combination with other sequences, for example, scaffold sequences from other species.

In another aspect, the heterologous partly feline immunoglobulin locus includes feline immunoglobulin V_{L} gene segment coding sequences, feline J_{L} gene segment coding sequences and non-coding sequences based on the non-coding sequences present in the endogenous L chain locus of the non-feline mammalian host cell genome. In one aspect, the non-coding sequences includes regulatory or scaffold sequences. In one aspect, the heterologous partly feline immunoglobulin locus is introduced into the host cell using recombinase targeting sites that have been previously introduced upstream of the endogenous immunoglobulin V_{L} gene locus and downstream of the endogenous J_{L} gene locus on the same chromosome.

In one aspect, the heterologous partly feline immunoglobulin locus is synthesized as a single nucleic acid and introduced into the non-feline mammalian host cell as a single nucleic acid region. The heterologous partly feline immunoglobulin locus may also be synthesized in two or more contiguous segments and introduced to the mammalian host cell as discrete segments. The heterologous partly feline immunoglobulin locus can also be produced using recombinant methods and isolated prior to being introduced into the non-feline mammalian host cell. In one aspect, a partly feline immunoglobulin heavy chain variable region locus can be generated in silico as follows: the genomic sequence of a mouse heavy chain immunoglobulin locus is obtained as well as feline V_{H}, D_{H} and J_{H} coding sequences, for example, from the National Center for Biotechnology Information or The International ImMunoGeneTics (IMGT) information system. The mouse V_{H}, D_{H} and J_{H} coding sequences are replaced in silico with feline V_{H}, D_{H} and J_{H} coding sequences, for example, using commercially available software. Advantageously, the V_{H}, D and J_{H} coding sequences can be replaced while leaving the intervening mouse non-coding sequences intact. Similarly, a partly feline immunoglobulin light chain variable region locus can be generated in silico as follows: the genomic sequence of a mouse light chain immunoglobulin locus is obtained as well as feline V_{L} and J_{L} coding sequences, for example, from the National Center for Biotechnology Information or The International ImMunoGeneTics (IMGT) information system. The mouse V_{L} and J_{L} coding sequences are replaced in silico with feline V_{L} and J_{L} coding sequences, for example, using commercially available software. Again, the V_{L} and J_{L} coding sequences can be replaced while leaving the intervening mouse non-coding sequences intact. Methods are known for synthesizing a DNA sequence that includes the partly feline immunoglobulin locus based on the in silico sequences.

In another aspect, a method is provided for generating a non-feline mammalian cell that includes a heterologous partly feline immunoglobulin locus. In one aspect, the method includes: a) introducing into the genome of a non-feline mammalian host cell two or more sequence-specific recombination sites that are not capable of recombining with one another, wherein at least one recombination site is introduced upstream of an endogenous immunoglobulin variable region gene locus and at least one recombination site is introduced downstream of the same endogenous immunoglobulin variable region gene locus; b) providing a vector that includes a heterologous partly feline immunoglobulin locus having i) feline immunoglobulin variable region gene coding sequences and ii) non-coding regulatory or scaffold sequences based on an endogenous immunoglobulin variable region gene locus of the host cell genome, wherein the partly feline immunoglobulin locus is flanked by the same two sequence-specific recombination sites that flank the endogenous immunoglobulin variable region gene locus of the host cell; c) introducing into the host cell the vector of step b) and a site specific recombinase capable of recognizing the two recombinase sites; d) allowing a recombination event to occur between the genome of the cell and the heterologous partly feline immunoglobulin locus, resulting in a replacement of the endogenous immunoglobulin variable region gene locus with the heterologous partly feline immunoglobulin variable region gene locus. In one aspect, the partly feline immunoglobulin locus includes feline V_{H} immunoglobulin gene segment coding sequences, and i) feline D_{H} and J_{H} gene segment coding sequences, ii) non-coding regulatory or scaffold sequences flanking individual V_{H}, D_{H}, and J_{H} gene segments present endogenously in the genome of the non-feline mammalian host, and iii) pre-D sequences based on the endogenous genome of the non-feline mammalian host cell. In one aspect, the recombinase targeting sites are introduced upstream of the endogenous immunoglobulin V_{H} gene locus and downstream of the endogenous J_{H} gene loci.

In one aspect, a transgenic rodent is provided with a genome in which a rodent endogenous immunoglobulin variable gene locus has been deleted and replaced with a heterologous partly feline immunoglobulin locus that includes feline immunoglobulin variable gene coding sequences and non-coding regulatory or scaffold sequences based on the rodent endogenous immunoglobulin variable gene locus. In one aspect, the heterologous partly feline immunoglobulin locus of the transgenic rodent is functional and expresses immunoglobulin chains that include feline variable domains and rodent constant domains. In one aspect, the heterologous partly feline immunoglobulin locus includes feline V_{H}, D_{H}, and J_{H} coding sequences. In one aspect, the heterologous partly feline immunoglobulin locus includes feline V_{L} and J_{L} coding sequences. In one aspect, the heterologous partly feline immunoglobulin locus includes feline kappa (κ) V_{L} and J_{L} coding sequences. In one aspect, the heterologous partly feline immunoglobulin locus includes feline lambda (λ) V_{L} and J_{L} coding sequences. In one aspect, a cell of B lymphocyte lineage from the transgenic rodent is provided. In one aspect, a part or whole immunoglobulin molecule that includes feline variable domain and rodent constant domain sequences obtained from the cell of B lymphocyte lineage are provided. In one aspect, a hybridoma cell derived from the cell of B lymphocyte lineage is provided. In one aspect, a part or whole immunoglobulin molecule that includes feline variable domains and rodent constant domains derived from the hybridoma cell is provided. In one aspect, an immortalized cell derived from the cell of B lymphocyte lineage is provided. In one aspect, a part or whole immunoglobulin molecule that includes feline variable domains and rodent constant domains derived from an immortalized cell is provided. In one aspect, a transgenic rodent is provided, wherein the heterologous partly feline immunoglobulin locus includes feline V_{L} and J_{L} coding sequences. In one aspect, a transgenic rodent is provided, in which the heterologous partly feline immunoglobulin loci includes feline V_{H}, D_{H}, and J_{H} coding sequences. In one aspect, the heterologous partly feline immunoglobulin locus includes feline kappa (κ) V_{L} and J_{L} coding sequences. In one aspect, the heterologous partly feline immunoglobulin locus includes feline lambda (λ) V_{L} and J_{L} coding sequences. In one aspect, the rodent is a mouse. In one aspect, the non-coding regulatory sequences include the one or more of the following sequences of the endogenous host: promoters preceding each V gene segment, splice sites, and recombination signal sequences for V(D)J recombination. In one aspect, the heterologous partly feline immunoglobulin locus further includes one or more of the following sequences of the endogenous host: ADAM6 gene, a Pax-5-Activated Intergenic Repeat (PAIR) elements, and CTCF binding sites from heavy chain intergenic control region 1 (IGCR1).

In one aspect, the non-feline cell is a mammalian cell. In one aspect, the non-feline mammalian cell is a mammalian embryonic stem (ES) cell.

In one aspect, non-feline mammalian cells in which the endogenous immunoglobulin variable region gene locus has been replaced with a heterologous partly feline immunoglobulin variable region gene locus are selected and isolated. In one aspect, the cells are non-feline mammalian ES cells, for example, rodent ES cells. In one aspect, at least one isolated non-feline mammalian cell is used to create a transgenic non-feline mammal expressing the heterologous partly feline immunoglobulin variable region gene loci. In one aspect, at least one isolated non-feline mammalian ES cell is used to create a transgenic non-feline mammal expressing the heterologous partly feline immunoglobulin variable region gene loci.

In one aspect, a method for generating the transgenic rodent is provided. In one aspect, the method includes: a) integrating at least one target site for a site-specific recombinase into the genome of a rodent cell upstream of an endogenous immunoglobulin variable gene locus and at least one target site for a site-specific recombinase downstream of the endogenous immunoglobulin variable gene locus. In one aspect, the endogenous immunoglobulin variable locus includes V_{H}, D_{H} and J_{H} gene segments. In one aspect, the endogenous immunoglobulin variable locus includes Vκ and Jκ gene segments. In one aspect, the endogenous immunoglobulin variable locus includes Vλ and Jλ gene segments. In one aspect, the endogenous immunoglobulin variable locus includes Vλ, Jλ gene segments and Cλ genes. In one aspect, the method includes: b) providing a vector that includes an heterologous partly feline immunoglobulin locus. In one aspect, said heterologous partly feline immunoglobulin locus includes chimeric feline immunoglobulin gene segments. In one aspect, each of the partly feline immunoglobulin gene segments include feline immunoglobulin variable gene coding sequences and rodent non-coding regulatory or scaffold sequences. In one aspect, the partly feline immunoglobulin variable gene locus is flanked by target sites for a site-specific recombinase. In one aspect, the target sites are capable of recombining with target sites introduced into the rodent cell. In one aspect, the method includes: c) introducing into the rodent cell the vector and a site-specific recombinase capable of recognizing the target sites. In one aspect, the method includes: d) allowing a recombination event to occur between the genome of the cell and the heterologous partly feline immunoglobulin locus, wherein the endogenous immunoglobulin variable gene locus is replaced with the heterologous partly feline immunoglobulin locus. In one aspect, the method includes: e) selecting a cell that includes the heterologous partly feline immunoglobulin variable locus generated in step d); and using the cell to create a transgenic rodent that includes the heterologous partly feline immunoglobulin variable locus. In one aspect, the cell is a rodent embryonic stem (ES) cell. In one aspect, the cell is a mouse embryonic stem (ES) cell.

In one aspect, the method further includes after step a) and before step b), a step of deleting the endogenous immunoglobulin variable gene locus by introducing a recombinase that recognizes a first set of target sites, wherein the deleting step leaves in place at least one set of target sites that are not capable of recombining with one another in the genome of the rodent cell. In one aspect, the vector includes feline V_{H}, D_{H}, and J_{H}, coding sequences. In one aspect, the vector includes feline V_{L} and J_{L} coding sequences. In one aspect, the heterologous partly feline immunoglobulin locus includes feline kappa (κ) V_{L} and J_{L} coding sequences. In one aspect, the heterologous partly feline immunoglobulin locus includes lambda (λ) V_{L} and J_{L} coding sequences. In one aspect, the vector further includes one or more of the following: a promoter, splice sites, and recombination signal sequences.

In one aspect, a method is provided for generating a transgenic non-feline mammal that includes a heterologous partly feline immunoglobulin variable region gene locus. In one aspect, the method includes: a) introducing into the genome of a non-feline mammalian host cell one or more sequence-specific recombination sites that flank an endogenous immunoglobulin variable region gene locus and are not capable of recombining with one another. In one aspect, the method includes: b) providing a vector that includes a partly feline immunoglobulin locus having i) feline variable region gene coding sequences and ii) non-coding regulatory or scaffold sequences based on the endogenous host immunoglobulin variable region gene locus. In one aspect, the coding and non-coding regulatory or scaffold sequences are flanked by the same sequence-specific recombination sites as those introduced to the genome of the host cell of a). In one aspect, the method includes: c) introducing into the cell the vector of step b) and a site-specific recombinase capable of recognizing one set of recombinase sites. In one aspect, the method includes: d) allowing a recombination event to occur between the genome of the cell of a) and the heterologous partly feline immunoglobulin variable region gene locus. In one aspect, the endogenous immunoglobulin variable region gene locus is replaced with the partly feline immunoglobulin locus. In one aspect, the method includes: e) selecting a cell that includes the partly feline immunoglobulin locus; and f) using the cell to create a transgenic mammal that includes the partly feline immunoglobulin locus.

In one aspect, the transgenic non-feline mammal is a rodent, e.g., a mouse or a rat.

In one aspect, an immunoglobulin library (also referred to as repertoire) is provided that includes a diversity of at least 10³ library members.

In one aspect, a repertoire of antibodies is provided that includes the partly feline antibody described herein. In one aspect, the repertoire includes a diversity of antibodies, that each specifically recognize the same target antigen. Such repertoire can be referred to as an antibody library of the same antibody type or structure, wherein antibodies differ in their antigen-binding sites, e.g., to produce antibody variants of a parent antibody recognizing the same epitope. In one aspect, the antibody library includes affinity matured or otherwise optimized antibody variants. In one aspect, the antibody library includes antibodies that specifically recognize a target antigen, but different epitopes of such target antigen.

In one aspect, the antibody repertoire is screened and individual library members are selected according to desired structural or functional properties, for example, to produce an antibody product.

In one aspect, a repertoire of antibodies is provided that include the partly feline antibody described herein. In one aspect, the repertoire includes a diversity of antibodies that recognize different target antigens. In one aspect, the repertoire is obtained by immunizing the non-feline mammal with multicomponent antigens, including, but not limited to, viruses or bacteria, which can have many different target antigens, each of which can include multiple epitopes.

In one aspect, the repertoire is a naive library of antibodies, which can also be referred to as a “pre-immune repertoire”. In one aspect, the pre-immune repertoire is expressed by mature but antigen-inexperienced B cells that have recently exited from the bone marrow.

In one aspect, the repertoire of antibodies can be characterized by a diversity encompassing at least about 10³ antibodies, for example, at least about 10⁴, about 10⁵, about 10⁶ or about 10⁷, each characterized by a different antigen-binding site.

In one aspect, a non-feline mammalian cell is provided that expresses a heterologous immunoglobulin variable region gene locus having feline variable region gene coding sequences and non-coding regulatory or scaffold sequences based on the endogenous non-feline immunoglobulin locus of the host genome. In one aspect, the non-feline mammalian cell expresses chimeric antibodies that include fully feline H or L chain variable domains in conjunction with their respective constant regions that are endogenous to the non-feline mammalian cell or mammal.

In one aspect, a non-feline transgenic mammal is provided that expresses a heterologous immunoglobulin variable region gene locus having feline variable region gene coding sequences and non-coding regulatory or scaffold sequences based on the endogenous non-feline immunoglobulin locus of the host genome. In one aspect, the non-feline transgenic mammal expresses chimeric antibodies that include fully feline H or L chain variable domains in conjunction with their respective constant regions that are endogenous to the non-feline mammalian cell or mammal.

In one aspect, B cells from transgenic non-feline mammals are provided that are capable of expressing partly feline antibodies having fully feline variable sequences. In one aspect, immortalized B cells are provided as a source of a monoclonal antibody specific for a particular antigen.

In one aspect, feline immunoglobulin variable region gene sequences are provided that are cloned from B cells for use in the production or optimization of antibodies for diagnostic, preventative and therapeutic uses.

In one aspect, non-feline hybridoma cells are provided that are capable of producing partly feline monoclonal antibodies having fully feline immunoglobulin variable region sequences.

In one aspect methods are provided for removing V_{H} and V_{L} exons that encode H and L chain immunoglobulin variable domains from monoclonal antibody-producing hybridomas and modifying the V_{H} and V_{L} exons to include feline constant regions, thereby creating a fully feline antibody that is not immunogenic when injected into cats.

In one aspect, a method of producing a feline antibody for therapeutic or diagnostic use is provided. In one aspect, the method includes:
(i) expressing an antibody with a feline variable domain cloned from an antibody-producing cell of a transgenic rodent whose genome includes an endogenous rodent immunoglobulin locus variable region that has been deleted and replaced with an heterologous immunoglobulin locus variable region that includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment that includes feline V, D or J immunoglobulin variable region coding sequences and rodent immunoglobulin variable region non-coding gene segment sequences; and
(ii) isolating the antibody with the feline variable domain, wherein the antibody is suitable for therapeutic or diagnostic use.

In one aspect, the antibody is cloned from a B cell of the transgenic rodent. In one aspect, the rodent is a mouse. In one aspect, a therapeutic or diagnostic antibody is provided that is produced by a method described herein.

In one aspect, a method of producing a therapeutic or diagnostic antibody with feline variable domains is provided. In one aspect, the method includes:
(i) cloning a feline variable domain of an antibody expressed by an antibody-producing cell from a transgenic rodent whose genome includes an endogenous rodent immunoglobulin locus variable region that has been deleted and replaced with an heterologous immunoglobulin locus variable region includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes feline V, D or J immunoglobulin variable region coding sequences and rodent immunoglobulin variable region non-coding gene segment sequences; and
(ii) producing the therapeutic or diagnostic antibody that includes the feline variable domain of the antibody expressed by the transgenic rodent.

In one aspect, the feline variable domain is cloned from an antibody expressed by a B cell from the transgenic rodent. In one aspect, the rodent is a mouse. In one aspect, a therapeutic or diagnostic antibody is provided that is produced by a method described herein.

In one aspect, a method is provided for producing a monoclonal antibody that includes a feline variable domain. In one aspect, the method includes:
(i) providing B cells from a transgenic rodent whose genome includes an endogenous rodent immunoglobulin locus variable region which has been deleted and replaced with an heterologous immunoglobulin locus variable region that includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes feline V, D or J immunoglobulin variable region coding sequences embedded in rodent immunoglobulin variable region non-coding gene segment sequences;
(ii) immortalizing the B cells; and
(iii) isolating monoclonal antibodies that include feline variable domains expressed by the immortalized B cells, or genes encoding the antibodies.

In one aspect, the method includes the steps of:
(iv) cloning the feline variable domains expressed by the B cells; and
(v) producing a therapeutic or diagnostic antibody that includes the feline variable domain cloned from the B cells of the transgenic rodent.

In one aspect, a method is provided for producing antibodies that include feline variable domains. In one aspect, the method includes providing a transgenic rodent whose genome includes an endogenous rodent immunoglobulin locus variable region which has been deleted and replaced with an heterologous immunoglobulin locus variable region that includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes feline V, D or J immunoglobulin variable region coding sequences embedded in rodent immunoglobulin variable region non-coding gene segment sequences, wherein the heterologous immunoglobulin locus of the transgenic rodent expresses antibodies that include feline variable domains.

In one aspect, the method includes isolating the antibodies with feline variable regions expressed by the transgenic rodent, or genes encoding the antibodies. In one aspect, the method includes: (i) obtaining B cells from the transgenic rodent expressing antibodies specific for the target antigen; (ii) immortalizing the B cells; and (iii) isolating antibodies specific for the target antigen from the immortalized B cells.

In one aspect, the method includes cloning feline variable regions from the B cells specific for the particular antigen. In one aspect, the rodent is a mouse. In one aspect, the method includes producing a therapeutic or diagnostic antibody using the feline variable regions cloned from the B cells. In one aspect, a therapeutic or diagnostic antibody is provided that is produced by the method described herein.

These and other aspects, are described in more detail below.

### BRIEF DESCRIPTION OF THE FIGURES

FIG. 1 depicts the mouse Igh locus (top) (including V (IghV), D (IghD), J (IghJ), and C (IghC) gene segments) located at the telomeric end of chromosome 12, the Igκ locus (middle) (including V (IgkV), J (IgkJ), and C (IgkC) gene segments) located on located on chromosome 6 and the Igλ locus (bottom) (including IglV (V), IglJ (J), and IglC (C) gene segments) located on chromosome 16. Also shown in the Igh locus are 1) PAIR elements, which are cis-regulatory sequences critical for Igh looping to ensure utilization of distal VH gene segments in VDJ rearrangements, 2) the Adam6a male fertility-enabling gene, 3) Intergenic Control Region 1 (IGCR1), which contains sites that regulate ordered, lineage-specific rearrangement of the Igh locus, 4) Eμ, the heavy chain intronic enhancer, 5) Sμ, the switch region, 6) the 3′ regulatory region (3′RR), a cis-acting element that controls isotype switching. Also shown in the Igκ locus are the 5′ (E5′) and 3′ (E3′) enhancers, and in the Igλ locus are three enhancers, Eλ 2-4, Eλ, and Eλ 3-1).

FIG. 2 is a schematic diagram illustrating the strategy of targeting by homologous recombination to introduce a first set of sequence-specific recombination sites into a region upstream of the H chain variable region gene locus in the genome of a non-feline mammalian host cell.

FIG. 3 is a schematic diagram illustrating the introduction of a second set of sequence-specific recombination sites into a region downstream of the H chain variable region gene locus in the genome of a non-feline mammalian cell via a homology targeting vector. The diagram also illustrates deletion of the endogenous immunoglobulin H chain variable region gene locus as well as the selectable markers from the genome of the non-feline mammalian host cell.

FIG. 4 is a schematic diagram illustrating the RMCE strategy to introduce an heterologous partly feline immunoglobulin H chain locus into the non-feline mammalian host cell genome that has been previously modified to delete the endogenous immunoglobulin H chain variable region locus.

FIG. 5 is a schematic diagram illustrating the introduction of an heterologous partly feline immunoglobulin κ L chain variable region gene locus into the endogenous immunoglobulin κ L chain locus of the mouse genome.

FIG. 6 is a schematic diagram illustrating the introduction of an heterologous partly feline immunoglobulin λ L chain variable region gene locus into the endogenous immunoglobulin λ L chain locus of the mouse genome.

### DEFINITIONS

The terms used herein are intended to have the plain and ordinary meaning as understood by those of ordinary skill in the art. The following definitions are intended to aid the reader in understanding the present invention but are not intended to vary or otherwise limit the meaning of such terms unless specifically indicated.

The term “locus” as used herein refers to a chromosomal segment or nucleic acid sequence that, respectively, is present endogenously in the genome or is (or about to be) introduced into the genome. For example, an immunoglobulin locus may include part or all of the genes (i.e., V_{H}, D_{H} and J_{H} gene segments or V_{L} and J_{L} gene segments as well as constant region genes) and intervening non-coding sequences (i.e., introns, enhancers, etc.) that support expression of immunoglobulin H or L chain polypeptides. The term “locus” (e.g., immunoglobulin heavy chain variable region locus) may refer to a specific portion of a larger locus (e.g., a portion of the immunoglobulin H chain locus that includes the V_{H}, D_{H} and J_{H} gene segments). Similarly, an immunoglobulin light chain variable region gene locus may refer to a specific portion of a larger locus (e.g., a portion of the immunoglobulin L chain locus that includes the V_{L} and J_{L} gene segments).

The term “immunoglobulin variable region gene” as used herein refers to a variable (V), diversity (D), joining (J) gene segment, including V_{H}, D_{H}, or J_{H} gene segments in the immunoglobulin heavy chain variable region or V_{L} or J_{L} gene segments in the immunoglobulin light chain variable region that encode a portion of an immunoglobulin H or L chain variable domain, respectively. The term “immunoglobulin variable region locus” as used herein refers to part of, or the entire, chromosomal segment or nucleic acid strand containing clusters of V_{H}, D_{H}, or J_{H} gene segments or V_{L} or J_{L} gene segments and the intervening non-coding sequences, including, for example, non-coding regulatory or scaffold sequences.

The term “gene segment” as used herein, refers to a nucleic acid sequence that encodes a part of the heavy chain or light chain variable domain of an immunoglobulin molecule. A gene segment can include coding and non-coding sequences. The coding sequence of a gene segment is a nucleic acid sequence that can be translated into a polypeptide, such as the leader peptide and the N-terminal portion of a heavy chain or light chain variable domain. The non-coding sequences of a gene segment are sequences flanking the coding sequence, which may include the promoter, 5′ untranslated sequence, intron intervening the coding sequences of the leader peptide, recombination signal sequence(s) (RSS), and splice sites. The gene segments in the immunoglobulin heavy chain (IGH) locus include the V_{H}, D_{H} and J_{H} gene segments (also referred to as IGHV, IGHD and IGHJ, respectively). The light chain variable region gene segments in the immunoglobulin κ and λ light loci can be referred to as V_{L} and J_{L} gene segments. In the κ light chain, the V_{L} and J_{L} gene segments can be referred to as V_{κ} and J_{κ} gene segments or IGKV and IGKJ. Similarly, in the λ light chain, the V_{L} and J_{L} gene segments can be referred to as V_{λ} and J_{λ} gene segments or IGLV and IGLJ.

The heavy chain constant region can be referred to as C_{H} or IGHC. The C_{H} region exons in the cat that encode IgM, IgD, IgG1a, IgG2, IgE, or IgA can be referred to as, respectively, C_{μ}, C_{δ}, C_{γ1a}, C_{γ2}, C_{ε} or C_{α}. Similarly, the immunoglobulin κ or λ constant region can be referred to as C_{κ} or C_{λ}, as well as IGKC or IGLC, respectively.

“Partly feline” as used herein refers to nucleic acids, or their expressed protein and RNA products, that include sequences corresponding to the sequences found in a given locus of both a feline and a non-feline mammalian host. “Partly feline” as used herein also refers to an immunoglobulin locus that includes nucleic acid sequences from both a feline and a non-feline mammal. In one aspect, “partly feline” refers to an immunoglobulin locus that includes, for example, nucleic acid sequences from a rodent, for example, a mouse. In one aspect, the partly feline nucleic acids have coding sequences of feline immunoglobulin H or L chain variable region gene segments and sequences based on the non-coding regulatory or scaffold sequences of the endogenous immunoglobulin locus of the non-feline mammal.

The term “based on” when used with reference to endogenous non-coding regulatory or scaffold sequences from a non-feline mammalian host cell genome refers to the non-coding regulatory or scaffold sequences that are present in the corresponding endogenous locus of the mammalian host cell genome. In one aspect, the term “based on” means that the non-coding regulatory or scaffold sequences that are present in the partly feline immunoglobulin locus share a relatively high degree of homology with the non-coding regulatory or scaffold sequences of the endogenous locus of the host mammal. In one aspect, the non-coding sequences in the partly feline immunoglobulin locus share at least about 80%, about 90%, about 95%, about 96%, about 97%, about 98%, about 99% or about 100% homology with the corresponding non-coding sequences found in the endogenous locus of the host mammal. In one aspect, the non-coding sequences in the partly feline immunoglobulin locus are the same as the corresponding non-coding sequences found in the endogenous locus of the host mammal. In one aspect, the non-coding sequences in the partly feline immunoglobulin locus are retained from an immunoglobulin locus of the host mammal. In one aspect, the non-coding sequences in the partly feline immunoglobulin locus are the same as the corresponding non-coding sequences present in the endogenous locus of the host mammal. In one aspect, the feline coding sequences are embedded in the non-regulatory or scaffold sequences of the immunoglobulin locus of the host mammal. In one aspect, the non-feline host animal is a rodent, such as a rat or mouse.

“Chimeric” refers to a nucleotide sequence that includes nucleotide sequences from two or more species of animal, or a polypeptide, for example, an antibody, encoded by a nucleotide sequence that includes nucleotide sequences from two or more species of animal. A “chimeric” immunoglobulin locus refers to an immunoglobulin locus that includes nucleic acid sequences from two or more species of animal. In one aspect, the chimeric immunoglobulin locus includes feline nucleic acid sequences and mouse nucleic acid sequences. In one aspect, the chimeric immunoglobulin includes protein sequences from two or more species of animal. In one aspect, the chimeric immunoglobulin includes feline sequences and mouse sequences. In one aspect, the chimeric immunoglobulin includes a feline variable domain and a mouse constant domain. In one aspect, the chimeric immunoglobulin variable region locus includes feline V_{H}, D_{H} and J_{H} coding sequences or feline V_{L} and J_{L} coding sequences and non-feline non-coding sequences. In one aspect, the chimeric immunoglobulin variable region locus includes feline V_{H}, D_{H} and J_{H} coding sequences or feline V_{L} and J_{L} coding sequences and mouse non-coding sequences.

“Flanking” as used herein, refers to a sequence, for example, a nucleotide sequence that is upstream or downstream to a reference sequence. In one aspect, the flanking sequence is adjacent to the reference sequence. In one aspect, a pair of sequences flank a reference sequence, such that a first sequence is upstream of the reference sequence and a second sequence is downstream of the reference sequence.

“Endogenous” refers to a nucleic acid sequence or polypeptide that is naturally occurring within an organism or cell.

“Heterologous” refers to a nucleic acid sequence or polypeptide that is not naturally occurring within an organism or cell.

“Non-coding regulatory sequences” refer to sequences that are known to be essential for (i) V(D)J recombination, (ii) isotype switching, (iii) proper expression of the full-length immunoglobulin H or L chains following V(D)J recombination, or (iv) alternate splicing to generate, e.g., membrane and secreted forms of the immunoglobulin H chain. “Non-coding regulatory sequences” may further include the following sequences: enhancer and locus control elements such as the CTCF and PAIR sequences (Proudhon, et al., Adv. Immunol. 128:123-182 (2015)); promoters preceding each endogenous V gene segment; splice sites; introns; or recombination signal sequences flanking each V, D, or J gene segment. In one aspect, the “non-coding regulatory sequences” of the partly feline immunoglobulin locus share at least about 70%, about 75%, about 80%, about 85%, about 90%, about 95%, about 96%, about 97%, about 98%, about 99% and up to about 100% homology with the corresponding non-coding sequences found in the endogenous immunoglobulin locus of the non-feline mammalian host cell. In one aspect, the “non-coding regulatory sequences” of the partly feline immunoglobulin locus have the same sequence as the corresponding non-coding sequences found in the endogenous immunoglobulin locus of the non-feline mammalian host cell.

“Scaffold sequences” refer to sequences intervening the gene segments present in the endogenous immunoglobulin locus of the host cell genome. In certain aspects, the scaffold sequences are interspersed by sequences essential for the expression of a functional non-immunoglobulin gene, for example, ADAM6A or ADAM6B. In one aspect, the scaffold sequences can include a naturally occurring nucleic acid sequence from another species. In one aspect, the scaffolding sequences can be heterologous, based on a naturally occurring nucleic acid sequence from another species. In one aspect, the scaffolding sequences can include an artificial sequence. In one aspect, the scaffold sequence includes sequences that are present in the immunoglobulin locus of the feline genome in combination with other sequences, for example, scaffold sequences from other species. The phrase “non-coding regulatory or scaffold sequence” is inclusive in meaning and can refer to both non-coding regulatory sequences and scaffold sequences in an immunoglobulin locus.

“Specifically binds” refers to the ability of an antibody or immunoglobulin to bind to an epitope or antigenic determinant of a particular antigen with a much higher affinity than the antibody or immunoglobulin binds to other antigens.

The term “homology targeting vector” refers to a nucleic acid sequence used to modify the endogenous genome of a mammalian host cell by homologous recombination. A homology targeting vector can include, for example, targeting sequences with homology to the corresponding endogenous sequences flanking a locus to be modified that is present in the genome of a non-feline mammalian host. In one aspect, the homology targeting vector includes at least one sequence-specific recombination site. In one aspect, the homology targeting vector includes non-coding regulatory or scaffold sequences. In one aspect, the homology targeting vector includes one or more selectable marker genes. In one aspect, the homology targeting vector can be used to introduce a sequence-specific recombination site into a particular region of a host cell genome.

“Site-specific recombination” or “sequence-specific recombination” refers to a process of DNA rearrangement between two compatible recombination sequences (also referred to as “sequence-specific recombination sites” or “site-specific recombination sequences”). Site-specific recombination can include any of the following three events: a) deletion of a preselected nucleic acid flanked by the recombination sites; b) inversion of the nucleotide sequence of a preselected nucleic acid flanked by the recombination sites, and c) reciprocal exchange of nucleic acid sequences proximate to recombination sites located on different nucleic acid strands. It is to be understood that this reciprocal exchange of nucleic acid segments can be exploited as a targeting strategy to introduce a heterologous nucleic acid sequence into the genome of a host cell.

The term “targeting sequence” refers to a sequence homologous to DNA sequences in the genome of a cell that flank or are adjacent to the region of an immunoglobulin locus to be modified. The flanking or adjacent sequence may be within the locus itself or upstream or downstream of coding sequences in the genome of the host cell. Targeting sequences are inserted into recombinant DNA vectors which can be used to transfect a host cell, for example, an ES cell, such that sequences to be inserted into the host cell genome, such as the sequence of a recombination site, are flanked by the targeting sequences of the vector.

The term “site-specific targeting vector” as used herein refers to a vector that includes a nucleic acid encoding a sequence-specific recombination site, an heterologous partly feline locus, and optionally a selectable marker gene. In one aspect, the “site-specific targeting vector” is used to modify an endogenous immunoglobulin locus in a host using recombinase-mediated site-specific recombination. The recombination site of the targeting vector is suitable for site-specific recombination with another corresponding recombination site that has been inserted into a genomic sequence of the host cell (e.g., via a homology targeting vector), adjacent to an immunoglobulin locus that is to be modified. Integration of a heterologous partly feline sequence into a recombination site in an immunoglobulin locus results in replacement of the endogenous locus by the heterologous partly feline region.

The term “transgene” is used herein to describe genetic material that has been or is about to be artificially inserted into the genome of a cell, and particularly a cell of a mammalian host animal. The term “transgene” as used herein refers to a partly feline nucleic acid, e.g., a partly feline nucleic acid in the form of a heterologous expression construct or a targeting vector.

“Transgenic animal” refers to a non-feline animal, usually a mammal, having an heterologous nucleic acid sequence present as an extrachromosomal element in a portion of its cells or stably integrated into its germ line DNA (i.e., in the genomic sequence of most or all of its cells). In the present invention, a partly feline nucleic acid is introduced into the germ line of such transgenic animals by genetic manipulation of, for example, embryos or embryonic stem cells of the host animal according to methods well known in the art.

A “vector” includes plasmids and viruses and any DNA or RNA molecule, whether self-replicating or not, that can be used to transform or transfect a cell.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach and Veksler, Eds. (2007), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Green and Sambrook (2012), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2021), Lehninger, Principles of Biochemistry 8e, W. H. Freeman Pub., New York, N.Y.; and Berg et al. (2019) Biochemistry, 93, Macmillan Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a locus” refers to one or more loci, and reference to “the method” includes reference to equivalent steps and methods known to those skilled in the art, and so forth.

As used herein, the term “or” can mean “and/or”, unless explicitly indicated to refer only to alternatives or the alternatives are mutually exclusive. The terms “including,” “includes” and “included” are not limiting.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well-known to those skilled in the art have not been described in order to avoid obscuring the invention.

In the humoral immune system, a diverse antibody repertoire is produced by combinatorial and junctional diversity of IGH and IGL chain gene loci by a process termed V(D)J recombination. In the developing B cell, the first recombination event to occur is between one D and one J gene segment of the heavy chain locus, and the DNA between these two gene segments is deleted. This D-J recombination is followed by the joining of one V gene segment from a region upstream of the newly formed DJ complex, forming a rearranged VDJ exon. All other sequences between the recombined V and D gene segments of the newly generated VDJ exon are deleted from the genome of the individual B cell. This rearranged exon is ultimately expressed on the B cell surface as the variable region of the H-chain polypeptide, which is associated with an L-chain polypeptide to form the B cell receptor (BCR). The murine and feline Ig loci are highly complex in the numbers of features they contain and in how their coding regions are diversified by V(D)J rearrangement; however, this complexity does not extend to the basic details of the structure of each variable region gene segment. The V, D and J gene segments are uniform in their compositions and organizations. For example, V gene segments have the following features that are arranged in essentially invariant sequential fashion in immunoglobulin loci: a short transcriptional promoter region (<600 bp in length), an exon encoding the majority of the signal peptide for the antibody chain; an intron; an exon encoding a small part of the signal peptide of the antibody chain and the majority of the antibody variable domain, and a 3′ recombination signal sequence necessary for V(D)J rearrangement. Similarly, D gene segments have the following features: a 5′ recombination signal sequence, a coding region and a 3′ recombination signal sequence. The J gene segments have the following features: a 5′ recombination signal sequence, a coding region and a 3′ splice donor sequence.

In one aspect, non-feline mammalian cells are provided that include a heterologous, partly feline nucleic acid sequence that includes feline variable region coding sequences and non-coding regulatory or scaffold sequences present in the immunoglobulin locus of the mammalian host genome, e.g., mouse genomic non-coding sequences when the host mammal is a mouse.

The feline genome V_{H} region includes approximately 24 V_{H} gene segments and 5 J_{H} gene segments. The number of D_{H} gene segments has not yet been precisely defined, but all gene segments map to feline chromosome B3 of the Abyssinian cat breed. The kappa (κ) coding region maps to feline chromosome A3, spanning about 200 kb, and contains approximately 12 functional Vκ, 5 Jκ and 1 Cκ genes. The lambda (λ) coding region maps to feline chromosome D3, spanning about 1000 kb, and contains approximately 32 functional Vλ, 10 functional Jλ and 12 Cλ genes, only 5 of which are functional. The feline IGL locus contains a high frequency (˜62/94) of apparently non-functional Vλ gene segments. In one aspect, in the partly feline H and κ and λ L chain loci, all V_{H}, D_{H} and J_{H} segments and all V_{L} and J_{L} segments are flanked by mouse RS S to promote rearrangement during B cell development and contribution to the partly feline antibody repertoire of the transgenic mouse.

As with humans and mice, cats express two types of Ig light chains (κ and λ). However, the κ to λ ratio differs significantly among these animals. In mice, approximately 96% of light chains in the serum antibodies are the κ type, while the κ type in humans accounts for only 66% of the total population of Ig L chains. In contrast, the L chain repertoire in cats is dominated (95%) by λ.

The partly feline nucleic acid sequences incorporated into the Igh, Igκ or Igλ loci allow the transgenic animal to produce antibodies that include feline heavy chain variable regions paired with feline κ or λ variable regions. The partly feline immunoglobulin variable region locus retains the regulatory sequences and other elements within the intervening sequences of the host genome (e.g., rodent) that help to promote efficient antibody production and antigen recognition in the host.

In one aspect, a synthetic, or recombinantly produced, partly feline immunoglobulin locus is provided that includes feline coding sequences and non-feline non-coding regulatory or scaffold sequences from an immunoglobulin V_{H}, Vλ or Vκ locus.

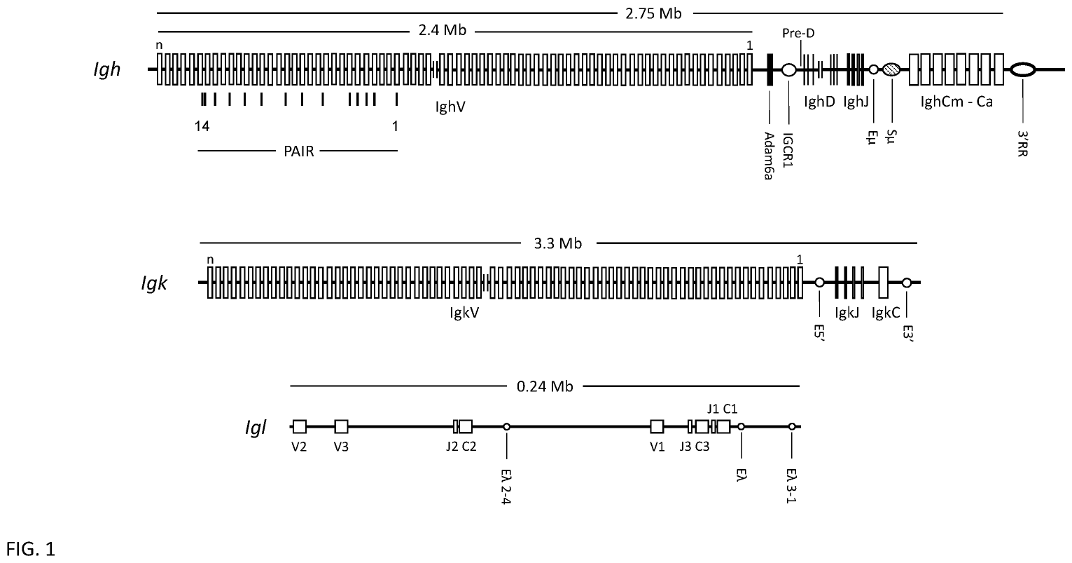

In one aspect the synthetic H chain DNA segment contains one or more of the following elements: the ADAM6 gene needed for male fertility, Pax-5-Activated Intergenic Repeats (PAIR) elements involved in Igh locus contraction, CTCF binding sites from the heavy chain intergenic control region 1, involved in regulating normal VDJ rearrangement [(Proudhon, et al., Adv. Immunol., 128:123-182 (2015)], or combinations thereof. The locations of these endogenous non-coding regulatory and scaffold sequences in the mouse Igh locus are depicted in FIG. 1, which illustrates from left to right: the ˜100 functional heavy chain variable region gene segments; PAIR, Pax-5 Activated Intergenic Repeats involved in Igh locus contraction for VDJ recombination; Adam6a, a disintegrin and metallopeptidase domain 6A gene required for male fertility; Pre-D region, a 21609 bp fragment upstream of the most distal D_{H} gene segment, Ighd-5; Intergenic Control Region 1 (IGCR1) that contains CTCF insulator sites to regulate V_{H} gene segment usage; D_{H}, diversity gene segments (10-15 depending on the mouse strain); four joining J_{H} gene segments; Eμ, the intronic enhancer involved in VDJ recombination; Sμ, the μ switch region for isotype switching; eight heavy chain constant region genes: Cμ, Cδ, Cγ3, Cγ1, Cγ2b, C2γa/c, Cε, and Cα; 3′ Regulatory Region (3′RR) that controls isotype switching and somatic hypermutation. FIG. 1 is modified from a figure taken from Proudhon, et al., Adv. Immunol., 128:123-182 (2015).

In one aspect, the heterologous partly feline immunoglobulin locus to be integrated into a mammalian host cell includes all or a substantial number of the known feline V_{H} gene segments. In some instances, however, it may be desirable to use a subset of such V_{H} gene segments. In one aspect, even as few as one feline V_{H} coding sequence may be included in the partly feline immunoglobulin locus.

In one aspect, the non-feline mammal or mammalian cell includes a heterologous partly feline immunoglobulin locus that includes feline V_{H}, D_{H}, and J_{H} gene coding sequences. In one aspect, the partly feline immunoglobulin locus includes non-coding regulatory and scaffold sequences, for example, pre-D sequences, based on the endogenous Igh locus of the non-feline mammalian host. In one aspect, the heterologous partly feline immunoglobulin locus includes a fully recombined V(D)J exon.

In one aspect, the transgenic non-feline mammal is a rodent, for example, a mouse, that includes a heterologous, partly feline immunoglobulin locus that includes feline V_{H}, D_{H}, and J_{H} genes and intervening sequences, including, for example, a pre-D region, based on the intervening (non-coding regulatory or scaffold) sequences in the rodent. In one aspect, the transgenic rodent further includes a partly feline IGL loci that include feline Vκ or Vλ coding sequences, and feline Jκ or Jλ coding sequences, respectively, and intervening sequences, such as non-coding regulatory or scaffold sequences present in the Igl loci of the rodent.

In one aspect, the entire endogenous V_{H} immunoglobulin locus of the mouse genome is deleted and replaced with 24 functional feline V_{H} gene segments and non-coding sequences of the J558 V_{H} locus of the mouse genome. In one aspect, the heterologous immunoglobulin locus includes feline D_{H} and 5 J_{H} gene segments. In one aspect, the heterologous immunoglobulin locus includes the mouse pre-D region. In one aspect, the feline V_{H}, D_{H}, and J_{H} coding sequences are embedded in the rodent non-coding sequences.

In one aspect, a combination of homologous recombination and site-specific recombination is used to generate transgenic cells and animals. In one aspect, a homology targeting vector is used to introduce sequence-specific recombination sites into a mammalian host cell genome at a desired location in the endogenous immunoglobulin loci. In one aspect, the sequence-specific recombination site is inserted into the genome of a mammalian host cell by homologous recombination and does not affect expression or coding sequences of any other genes in the mammalian host cell. In one aspect, the ability of the immunoglobulin genes to be transcribed and translated to produce antibodies is maintained after the recombination sites and, optionally, any additional sequence such as a selectable marker gene are inserted. However, in some cases it is possible to insert other heterologous sequences into an immunoglobulin locus sequence such that an amino acid sequence of the resultant antibody molecule is altered by the insertion, but the antibody retains sufficient functionality for the desired purpose. In one aspect, one or more polymorphisms are introduced into the endogenous locus in the constant region exons, thereby providing an allotypic marker so that the different Ig alleles can be distinguished.

In one aspect, the homology targeting vector is used to replace sequences within the endogenous immunoglobulin locus as well as to insert sequence-specific recombination sites and one or more selectable marker genes into the host cell genome. It is understood by those of ordinary skill in the art that a selectable marker gene as used herein can be exploited to identify and eliminate cells that have not undergone homologous recombination or cells that harbor random integration of the targeting vector.

Methods for homologous recombination are known and include those described in U.S. Pat. Nos. 6,689,610; 6,204,061; 5,631,153; 5,627,059; 5,487,992; and 5,464,764, each of which is incorporated by reference in its entirety.

### Site/Sequence-Specific Recombination

Site/sequence-specific recombination differs from homologous recombination in that short, specific DNA sequences, which are required for recognition by a recombinase, are the only sites at which recombination occurs. Depending on the orientations of these sites on a particular DNA strand or chromosome, the specialized recombinases that recognize these specific sequences can catalyze i) DNA excision or ii) DNA inversion or rotation. Site-specific recombination can also occur between two DNA strands if these sites are not present on the same chromosome. A number of bacteriophage- and yeast-derived site-specific recombination systems, each including a recombinase and its cognate recognition sites, have been shown to work in eukaryotic cells, including, but not limited to, the bacteriophage P1 Cre/lox system, the yeast FLP-FRT system, and the Dre system of the tyrosine family of site-specific recombinases. Such systems and methods are described, e.g., in U.S. Pat. Nos. 7,422,889; 7,112,715; 6,956,146; 6,774,279; 5,677,177; 5,885,836; 5,654,182; and 4,959,317, each of which is incorporated herein by reference.

Other systems of the tyrosine family of site-specific recombinases can be used, including, but not limited to, bacteriophage lambda integrase, HK2022 integrase, and systems belonging to the serine family of recombinases, including, for example, bacteriophage phiC31, and R4Tp901 integrases.

Because site-specific recombination can occur between two different DNA strands, site-specific recombination can be used to introduce a heterologous immunoglobulin locus into a host cell genome by a process called recombinase-mediated cassette exchange (RMCE). The RMCE process can be exploited using wild-type and mutant sequence-specific recombination sites for a recombinase protein. For example, a chromosomal locus to be targeted may be flanked by a wild-type LoxP site on one end and by a mutant LoxP site on the other. Likewise, a vector can include a heterologous sequence to be inserted into the host cell genome that is flanked by a wild-type LoxP site on one end and by a mutant LoxP site on the other. When the vector is transfected into the host cell in the presence of Cre recombinase, Cre recombinase will catalyze RMCE between the endogenous DNA strands and the DNA of the vector, rather than catalyzing an excision reaction on the same DNA strands, because the wild-type LoxP and mutant LoxP sites on each DNA strand are incompatible for recombination with each other. As such, the LoxP site on one DNA strand will only recombine with a LoxP site on the other DNA strand; and similarly, the mutated LoxP site on one DNA strand will only recombine with a mutated LoxP site on the other DNA strand.

In one aspect, variants of the sequence-specific recombination sites that are recognized by the same recombinase for RMCE are used. Examples of such sequence-specific recombination site variants include those that contain a combination of inverted repeats or those that include recombination sites with mutant spacer sequences. For example, two classes of variant recombinase sites are available to engineer stable Cre-loxP integrative recombination. Both exploit sequence mutations in the Cre recognition sequence, either within the 8 bp spacer region or the 13-bp inverted repeats. Spacer mutants such as lox511 [Hoess, et al., Nucleic Acids Res, 14:2287-2300 (1986)], lox5171 and lox2272 [Lee and Saito, Gene, 216:55-65 (1998)], m2, m3, m7, and m11 [Langer, et al., Nucleic Acids Res, 30:3067-3077 (2002)] recombine readily with themselves but have a markedly reduced rate of recombination with the wild-type site. This class of mutants has been exploited for DNA insertion by RMCE using non-interacting Cre-Lox recombination sites and non-interacting FLP recombination sites [Baer and Bode, Curr Opin Biotechnol, 12:473-480 (2001); Albert, et al., Plant J, 7:649-659 (1995); Seibler and Bode, Biochemistry, 36:1740-1747 (1997); Schlake and Bode, Biochemistry, 33:12746-12751 (1994)].

Inverted repeat mutants are another class of variant recombinase sites. For example, LoxP sites can contain altered bases in the left inverted repeat (LE mutant) or the right inverted repeat (RE mutant). An LE mutant, lox71, has 5 bp on the 5′ end of the left inverted repeat that is changed from the wild type sequence to TACCG [Araki, et al, Nucleic Acids Res, 25:868-872 (1997)]. Similarly, the RE mutant, 1ox66, has the five 3′-most bases changed to CGGTA. Inverted repeat mutants are used for integrating plasmid inserts into chromosomal DNA with the LE mutant designated as the “target” chromosomal loxP site into which the “donor” RE mutant recombines. Post-recombination, loxP sites are located in cis, flanking the inserted segment. The mechanism of recombination is such that, post-recombination, one loxP site is a double mutant (containing both the LE and RE inverted repeat mutations) and the other is wild type [Lee and Sadowski, Prog Nucleic Acid Res Mol Biol, 80:1-42 (2005); Lee and Sadowski, J Mol Biol, 326:397-412 (2003)]. The double mutant is sufficiently different from the wild-type site that it is unrecognized by Cre recombinase and the inserted segment is not excised.

In one aspect, sequence-specific recombination sites are introduced into introns, rather than coding or regulatory sequences to avoid disrupting regulatory sequences or coding sequences used in antibody expression.

Introduction of the sequence-specific recombination sites may be achieved by conventional homologous recombination techniques. Such techniques are described in references such as e.g., Green and Sambrook (2012) (Molecular cloning: a laboratory manual 4th ed. (Cold Spring Harbor, N.Y.: Cold Spring Harbor Laboratory Press) and Nagy, A. (2003). (Manipulating the mouse embryo: a laboratory manual, 3rd ed. (Cold Spring Harbor, N.Y.: Cold Spring Harbor Laboratory Press).

Specific recombination into the genome can be facilitated using vectors designed for positive or negative selection as known in the art. In order to facilitate identification of cells that have undergone the replacement reaction, an appropriate genetic marker system may be employed and cells selected by, for example, use of a selection tissue culture medium. In one aspect, nucleic acid sequences at or adjacent to the two end points of the heterologous sequence, for example, a marker system or gene can be removed following selection of the cells containing the heterologous nucleic acid.

In one aspect, cells in which the endogenous immunoglobulin locus has been deleted may be positively selected for using a marker gene, which can optionally be removed from the cells following or as a result of the recombination event. A positive selection system that may be used is based on the use of two non-functional portions of a marker gene, such as Hypoxanthine-guanine phosphoribosyltransferase (HPRT), that are brought together through the recombination event. In one aspect, the two non-functional portions are brought into functional association upon a successful replacement of the endogenous immunoglobulin locus with the heterologous immunoglobulin locus. In one aspect, the functionally reconstituted marker gene is flanked on either side by further sequence-specific recombination sites (which are different from the sequence-specific recombination sites used for the replacement reaction), such that the marker gene can be excised from the genome, using an appropriate site-specific recombinase. In another aspect, cells are negatively selected against upon exposure to a toxin or drug. For example, cells in which a targeting construct is not integrated by homologous recombination but is randomly integrated into the genome will retain expression of Herpes Simplex Virus-Thymidine Kinase (HSV-TK) if the HSV-TK gene is located outside of the region of homology. Such cells can be selected against using nucleoside analogues such as ganciclovir.

In one aspect, the recombinase is provided as a purified protein. In one aspect, the recombinase is provided as a protein expressed from a vector construct transiently transfected into the host cell or stably integrated into the host cell genome. Alternatively, a transgenic animal that includes the heterologous immunoglobulin locus may be crossed with an animal that expresses the recombinase.

In one aspect, two or more sets of sequence-specific recombination sites are included within the engineered genome, such that multiple rounds of RMCE can be exploited to insert the partly feline immunoglobulin variable region locus into a non-feline mammalian host cell genome.

In one aspect, the partly feline immunoglobulin locus is introduced using CRISPR technology. For example, the CRISPR/Cas9 genome editing system may be used for targeted recombination [He, et al., Nuc. Acids Res., 44:e85, (2016)].

### Generation of Transgenic Animals

In one aspect, methods are provided for the creation of transgenic animals, for example, rodents, for example, mice, that include a heterologous partly feline immunoglobulin locus.

In one aspect, the genome of the transgenic animal is modified so that B cells of the transgenic animal are capable of expressing more than one functional VH domain per cell, i.e., the cells produce bispecific antibodies as described in WO20170/35252, filed Aug. 24, 2016, entitled “Enhanced Production of Immunoglobulins”, the disclosure of which is incorporated by reference herein.

In one aspect, the genome of the transgenic animal is modified so that B cells of the transgenic animal are capable of expressing antibodies that include heavy chains but no light chains, i.e., the cells produce heavy chain-only antibodies.

In one aspect, the host cell is an embryonic stem (ES) cell, which can then be used to create a transgenic mammal. In one aspect, the method includes: isolating an embryonic stem cell that includes the heterologous partly feline immunoglobulin locus and using the ES cell to generate a transgenic animal that contains the heterologous partly feline immunoglobulin locus.

### EXAMPLES

The following examples are put forth to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations or modifications may be made without departing from the spirit or scope of the invention as described herein. The examples are, therefore, to be considered as illustrative and not restrictive.

Efforts have been made to ensure accuracy with respect to terms and numbers used (e.g., vectors, amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

The examples illustrate targeting by both a 5′ vector and a 3′ vector that flank a site of recombination and introduction of synthetic DNA via RMCE. Upon reading the specification, it will be apparent to one skilled in art that the 5′ vector targeting can take place first followed by the 3′, or the 3′ vector targeting can take place first followed by the 5′ vector. In some circumstances, targeting can be carried out simultaneously with dual detection mechanisms. Although some different strategies are used in each example to select for cells that have properly integrated the 5′ or 3′ vector, it will also be apparent that, with minor modifications, such strategies are interchangeable for targeting the Igh, Igκ or Igλ loci.

Example 1: Introduction of an Heterologous Partly Feline Immunoglobulin Variable Region Gene Locus into the Immunoglobulin H Chain Variable Region Gene Locus of a Non-Feline Mammalian Host Cell Genome

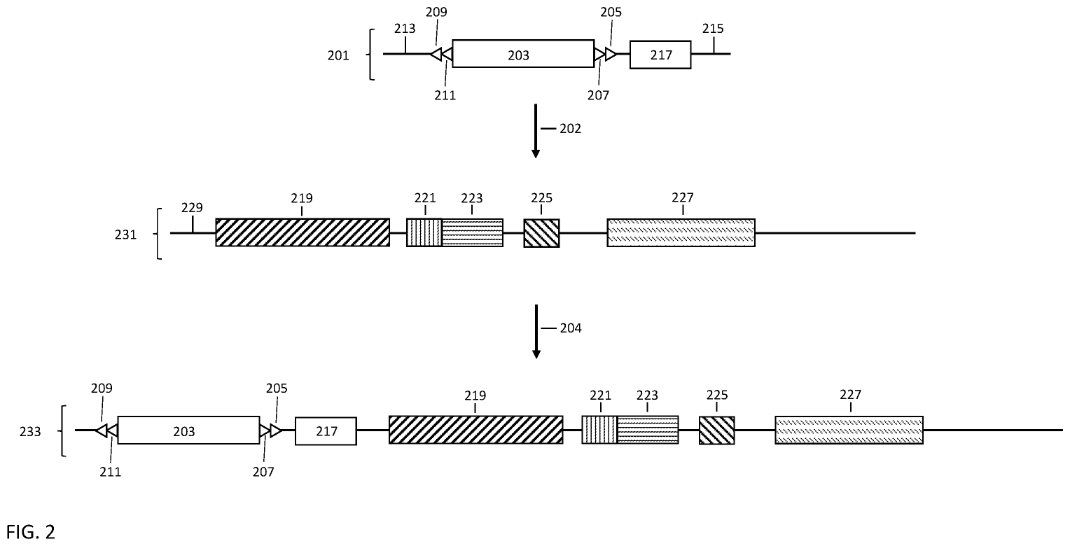

An exemplary method illustrating the introduction of a heterologous partly feline immunoglobulin locus into the genomic locus of a non-mammalian ES cell is illustrated in FIGS. 2-4. FIG. 2 depicts a method for introducing site-specific recombination sequences upstream (5′) of the endogenous V_{H} gene segments. A 5′ homology targeting vector (201) is provided that includes a puromycin phosphotransferase-thymidine kinase fusion protein (puro-TK) (203) flanked by two different recombinase recognition sites (e.g., FRT (207) and loxP (205) for Flp and Cre, respectively) and two different mutant sites (e.g., modified mutant FRT (209) and mutant loxP (211)) that lack the ability to recombine with their respective wild-type counterparts/sites (i.e., wild-type FRT (207) and wild-type loxP (205)). The targeting vector includes a diphtheria toxin receptor (DTR) cDNA (217) for use in negative selection of cells. The targeting vector also optionally includes a visual marker such as a green fluorescent protein (GFP) (not shown). The regions 213 and 215 are homologous to the 5′ and 3′ portions, respectively, of a contiguous region (229) in the endogenous non-feline locus that is 5′ of the genomic region that includes the endogenous non-feline V_{H} gene segments (219). The homology targeting vector (201) is introduced (202) into the ES cell, which has an immunoglobulin locus (231) that includes endogenous V_{H} gene segments (219), the pre-D region (221), the D_{H} gene segments (223), J_{H} gene segments (225), and the immunoglobulin constant gene region genes (227). The site-specific recombination sequences and the DTR cDNA from the homology targeting vector (201) are integrated (204) into the non-feline genome at a site 5′ of the endogenous mouse V_{H} gene locus, resulting in the genomic structure illustrated at 233.

Mouse embryonic stem (ES) cells (derived from C57B1/6NTac mice) are transfected by electroporation with the 5′ vector (201) according to known procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under selection for cells that have integrated the 5′ vector into their DNA. The ES cells that do not have the 5′ vector (201) integrated into their genome can be selected against (killed) by including puromycin in the culture medium; only the ES cells that have stably integrated the 5′ vector (201) into their genome and constitutively express the puro-TK gene are resistant to puromycin.

Colonies of drug-resistant ES cells are physically extracted from their plates after they became visible to the naked eye about a week later. These picked colonies are disaggregated, re-plated in micro-well plates, and cultured for several days. Thereafter, each of the clones of cells is divided such that some of the cells can be frozen as an archive, and the rest used for isolation of DNA for analytical purposes. The primary screening procedure for the introduction of 5′ vector can be carried out by Southern blotting, or by PCR with confirmations from secondary screening methods such as Southern blotting.

DNA from the ES cell clones is screened by PCR using a widely practiced gene-targeting assay design. For this assay, one of the PCR oligonucleotide primer sequences maps outside the region of identity shared between the 5′ vector (201) and the genomic DNA, while the other maps within the 5′ vector, e.g., in the Puro-TK gene (203). According to the standard design, these assays detect DNA that would only be present in clones of ES cells that undergo homologous recombination between the 5′ targeting vector and the endogenous mouse Igh locus.

The Southern blot assays are performed according to widely used procedures using three probes and genomic DNA digested with multiple restriction enzymes chosen so that the combination of probes and digests allow the structure of the targeted locus in the clones to be identified as properly modified by homologous recombination. One of the probes maps to DNA sequence flanking the 5′ side of the region of identity shared between the 5′ targeting vector and the genomic DNA; a second probe maps outside the region of identity but on the 3′ side; and the third probe maps within the novel DNA between the two arms of genomic identity in the vector, e.g., in the Puro-TK gene (203). The Southern blot identifies the presence of the expected restriction enzyme-generated fragment of DNA corresponding to the modified sequence, i.e., by homologous recombination with the 5′ targeting vector, part of the Igh locus as detected by one of the external probes and by the Puro-TK probe. The external probe detects the mutant fragment and also a wild-type fragment from the non-mutant copy of the immunoglobulin Igh locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. ES cell clones that have the expected genomic structure based on the Southern blot data, and that do not have detectable chromosomal aberrations based on the karyotype analysis, are selected for further use.

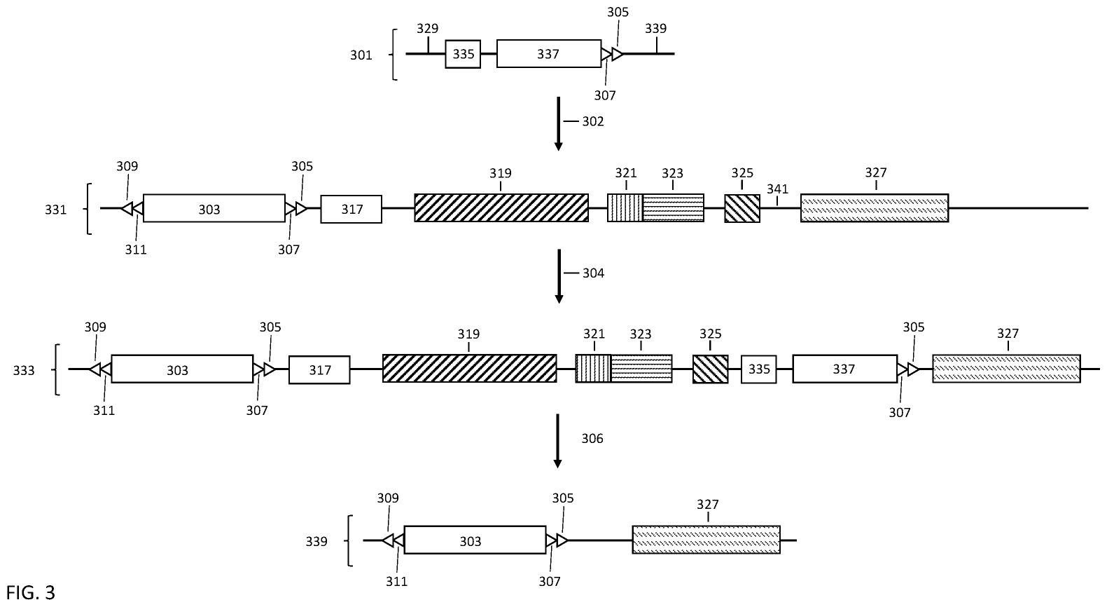

As illustrated in FIG. 3, a 3′ homology targeting vector (301) is provided that includes an optional hypoxanthine-guanine phosphoribosyltransferase (HPRT) gene (335) that can be used for positive selection in HPRT-deficient ES cells; a neomycin resistance gene (337); recombinase recognition sites FRT (307) and loxP (305), for Flp and Cre, respectively. The regions 329 and 339 are homologous to the 5′ and 3′ portions, respectively, of a contiguous region (341) in the endogenous mouse locus that is downstream of the endogenous J_{H} gene segments (325) and upstream of the constant region genes (327). The homology targeting vector is introduced (302) into the modified mouse immunoglobulin locus (331), which includes the endogenous V_{H} gene segments (319), the pre-D region (321), the D_{H} gene segments (323), the J_{H} gene segments (325), and the constant region genes (327). The site-specific recombination sequences (307, 305), the HPRT gene (335) and a neomycin resistance gene (337) of the homology targeting vector are integrated (304) into the mouse genome upstream of the endogenous mouse constant region genes (327), resulting in the genomic structure illustrated at 333.

Acceptable clones modified with the 3′ vector (301) are identified using procedures and screening assays that are essentially identical in design to those used with the 5′ vector (201) except that neomycin or HPRT selection is used instead of puromycin for selection. The PCR assays, probes and digests are also tailored to match the genomic region modified by the 3′ vector. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use.

Clones of ES cells that have been mutated by both the 3′ and the 5′ vectors, i.e., doubly targeted cells carrying both engineered mutations, are isolated following vector targeting and analysis. The clones must have undergone gene targeting on the same chromosome, as opposed to homologous chromosomes (i.e., the engineered mutations created by the targeting vectors must be in cis on the same DNA strand rather than in trans on separate homologous DNA strands). Clones with the cis arrangement are distinguished from those with the trans arrangement by analytical procedures such as fluorescence in situ hybridization of metaphase spreads using probes that hybridize to the novel DNA present in the two gene targeting vectors (303 and 337) between their arms of genomic identity. The two types of clones can also be distinguished from one another by transfecting them with a vector expressing Cre recombinase, which deletes the HPRT (335) and neomycin resistance (337) genes if the targeting vectors have been integrated in cis, and then analyzing the drug resistance phenotype of the clones by a “sibling selection” screening procedure in which some of the cells from each clone are tested for resistance to G418/neomycin. The majority of the resulting cis-derived clones are also sensitive to G418/neomycin, in contrast to the trans-derived clones, which should retain resistance to the drugs. Doubly targeted clones of cells with the cis-arrangement of engineered mutations in the heavy chain locus are selected for further use.

Once the two recombination sites are integrated into the mammalian host cell genome, the endogenous immunoglobulin locus is then subjected to recombination by introducing one of the recombinases corresponding to the sequence-specific recombination sites integrated into the genome, e.g., either Flp or Cre. In the presence of Flp or Cre (306), all the intervening sequences between the wild-type FRT or wild-type LoxP sites including the DTR gene (317), the endogenous Igh variable region gene loci (319, 323, 325), the pre-D region (321), and the HPRT (335) and neomycin resistance (337) genes are deleted, resulting in a genomic structure illustrated at 339. The procedure depends on the second targeting having occurred on the same chromosome rather than on its homolog (i.e., in cis rather than in trans). If the targeting occurs in cis as intended, the cells are not sensitive to negative selection by diphtheria toxin introduced into the media, because the DTR gene (317) that causes sensitivity to diphtheria toxin should be absent (deleted) from the host cell genome. Likewise, ES cells that harbor random integration of the first or second targeting vector(s) are rendered sensitive to diphtheria toxin by presence of the undeleted DTR gene.

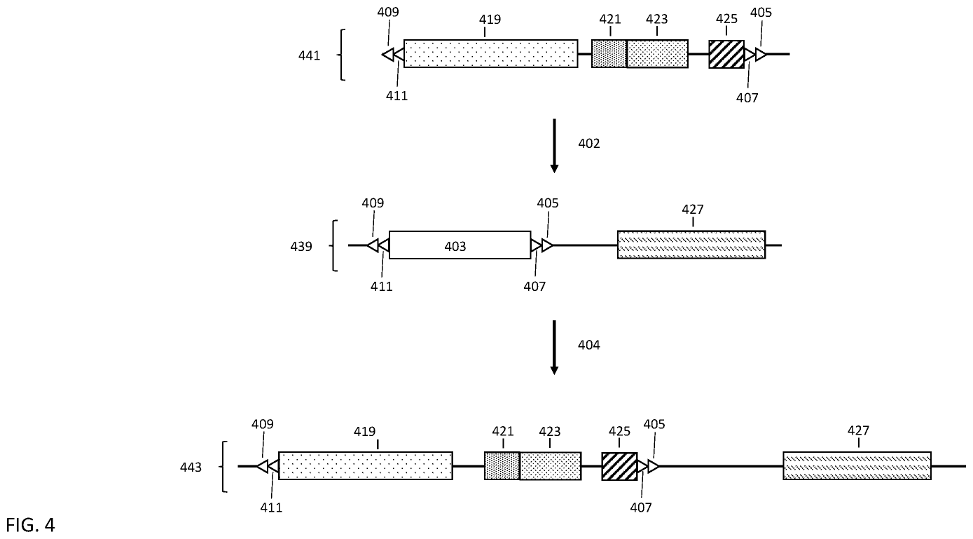

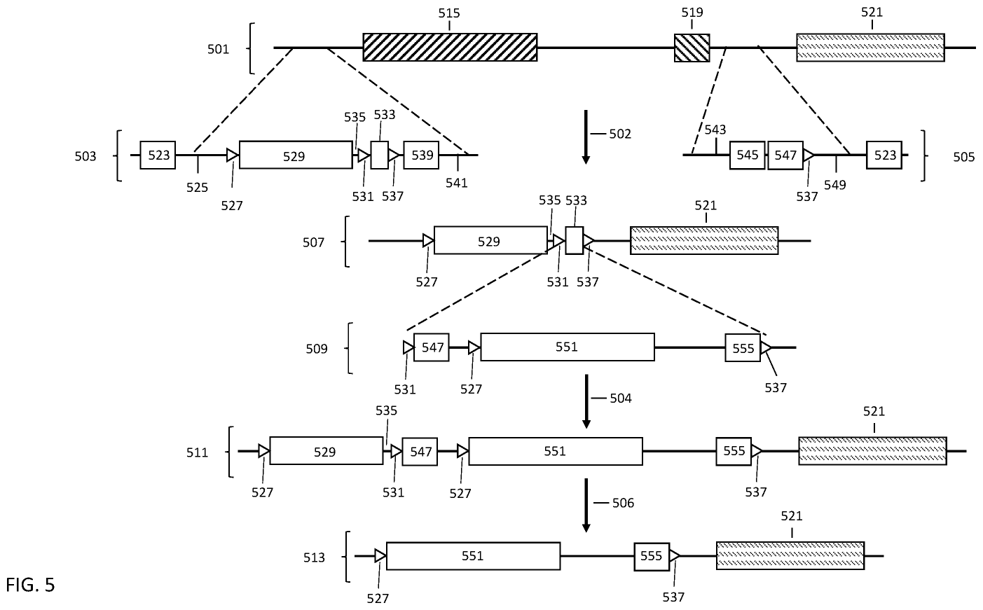

ES cell clones carrying the sequence deletion in one of the two homologous copies of their immunoglobulin heavy chain locus are retransfected with a Cre recombinase expression vector and a vector that includes a partly feline immunoglobulin heavy chain locus containing feline V_{H}, D_{H} and J_{H} gene segment coding sequences embedded in mouse non-coding sequences. FIG. 4 illustrates introduction of the heterologous partly feline immunoglobulin heavy chain locus into a mouse genome in which the part of the endogenous immunoglobulin heavy chain locus that encodes the heavy chain variable region domains has been deleted, including the intervening sequences between the endogenous V_{H} and J_{H} gene loci. A site-specific targeting vector (441) that includes a partly feline immunoglobulin locus to be inserted into the non-feline host genome is introduced (402) into the modified genome of the host cell (439) by RMCE. The site-specific targeting vector (441) that includes a partly feline V_{H} gene locus (419), mouse pre-D region (421), a partly feline D_{H} gene locus (423), a partly feline J_{H} gene locus (425), as well as flanking mutant FRT (409), mutant LoxP (lox5171; 411) wild-type FRT (407) and wild-type LoxP (405) sites is introduced (402) into the host cell by RMCE. Specifically, the partly feline V_{H} gene locus (419) includes 24 functional feline V_{H} gene segment coding sequences and 3′ non-feline RSS and intervening sequences present in the endogenous non-feline genome; the pre-D region (421) includes a 21.6 kb non-feline sequence present upstream in the endogenous non-feline genome; the D_{H} region (423) includes codons of feline D_{H} gene segments flanked by non-feline RSS and embedded in the intervening sequences present in the endogenous non-feline D_{H} region; and the J_{H} gene locus (425) includes codons of 5 feline J_{H} gene segments with 5′ non-feline RSS and embedded in the intervening sequences present in the endogenous non-feline genome. In one aspect, the Igh locus of the host cell genome is modified to delete all endogenous V_{H}, D_{H}, and J_{H} gene segments including the intervening sequences as described in relation to FIG. 3. As a consequence of this modification, the endogenous non-feline Igh locus (439) is left with a puro-TK fusion gene (403), which is flanked by a mutant FRT site (409) and a mutant LoxP site (lox5171; 411) upstream as well as a wild-type FRT (407) and a wild-type LoxP (405) downstream. Upon introduction of the appropriate recombinase (404), the partly feline immunoglobulin locus is integrated between the lox5171 (411) and wild-type loxP (405) sites into the genome upstream of the endogenous mouse constant region genes (427), to create the DNA region illustrated at 443.

ES cells that have not undergone RMCE and integration of the partly feline Igh locus retain the puro-TK fusion gene (403) and are eliminated by inclusion of ganciclovir to the tissue culture media.

The sequences of currently annotated functional feline V_{H}, D_{H} and J_{H} gene segments are shown in SEQ ID NO. 1-13. As the feline IGH locus is not fully annotated, additional gene segments exist that can be used in the animals, cells and methods described herein.

Integration of the heterologous partly feline immunoglobulin region can be detected by Southern blotting, or by PCR with confirmations from secondary screening methods such as Southern blotting. The screening methods are designed to detect the presence of the inserted V_{H}, D_{H} or J_{H} gene loci, as well as the intervening sequences. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use

ES cell clones carrying the partly feline immunoglobulin heavy chain variable region (443) in the mouse heavy chain locus are microinjected into mouse blastocysts from strain DBA/2 to create ES cell-derived chimeric mice according to standard procedures. Male chimeric mice with the highest levels of ES cell-derived contribution to their coats are selected for mating to female mice. Offspring from these matings are analyzed for the presence of the partly feline immunoglobulin heavy chain locus. Mice that carry the partly feline immunoglobulin heavy chain locus are used to establish a colony of mice.

### Example 2: Introduction of an Heterologous Partly Feline Immunoglobulin Locus into the Immunoglobulin Kappa Chain Gene Locus of a Mouse Genome

A method for replacing a portion of a mouse Igκ locus with partly feline Igκ locus is illustrated in FIG. 5. This method includes introducing a first site-specific recombinase recognition sequence into the mouse genome, which may be introduced either 5′ or 3′ of the cluster of endogenous V_{K} (515) and J_{K} (519) region gene segments of the mouse genome, followed by the introduction of a second site-specific recombinase recognition sequence into the mouse genome, which in combination with the first sequence-specific recombination site, flanks the entire locus that includes clusters of V_{K} and J_{K} gene segments upstream of the constant region gene (521). The flanked region is deleted and replaced with a partly feline immunoglobulin light chain variable region locus using the relevant site-specific recombinase.

The targeting vectors employed for introducing the site-specific recombination sequences on either side of the V_{K} (515) and J_{K} (519) gene segments also include an additional site-specific recombination sequence that is modified so that it is still recognized efficiently by the recombinase but does not recombine with unmodified sites. This site is positioned in the targeting vector such that after deletion of the V_{K} and J_{K} gene segment clusters it can be used for a second site specific recombination event in which a heterologous immunoglobulin light chain variable region locus is inserted into the modified V_{K} locus via RMCE. In this example, the heterologous immunoglobulin light chain variable region locus is a synthetic nucleic acid that includes feline V_{K} and J_{K} gene segments and mouse Igκ variable region non-coding sequences.

Two gene targeting vectors are constructed to accomplish the process just outlined. One of the vectors (503) includes mouse genomic DNA (525 and 541) taken from the 5′ end of the locus, upstream of the most distal V_{K} gene segment (515). The other vector (505) includes mouse genomic DNA (543 and 549) taken from within the locus downstream (3′) of the J_{K} gene segments (519) and upstream of the constant region gene (521).

The key features of the 5′ vector (503) are as follows: a gene encoding the diphtheria toxin A subunit (DTA) under transcriptional control of a modified herpes simplex virus type I thymidine kinase gene promoter coupled to two mutant transcriptional enhancers from the polyoma virus (523); 6 Kb of mouse genomic DNA (525) mapping upstream of the most distal variable region gene in the kappa chain locus; a FRT recognition sequence for the Flp recombinase (527); a piece of genomic DNA containing the mouse Polr2a gene promoter (529); a translation initiation sequence (535, methionine codon embedded in a “Kozak” consensus sequence); a mutated loxP recognition sequence (lox5171) for the Cre recombinase (531); a transcription termination/polyadenylation sequence (533); a loxP recognition sequence for the Cre recombinase (537); a gene encoding a fusion protein included of a protein conferring resistance to puromycin fused to a truncated form of the thymidine kinase (pu-TK) under transcriptional control of the promoter from the mouse phosphoglycerate kinase 1 gene (539); 2.5 Kb of mouse genomic DNA (541) mapping close to the 6 Kb sequence at the 5′ end in the vector and arranged in the native relative orientation.

The key features of the 3′ vector (505) are as follows: 6 Kb of mouse genomic DNA (543) mapping within the intron between the J_{κ} (519) and C_{κ} (521) gene loci; a gene encoding the human hypoxanthine-guanine phosphoribosyl transferase (HPRT) under transcriptional control of the mouse Polr2a gene promoter (545); a neomycin resistance gene under the control of the mouse phosphoglycerate kinase 1 gene promoter (547); a loxP recognition sequence for the Cre recombinase (537); 3.6 Kb of mouse genomic DNA (549) that maps immediately downstream in the genome of the 6 Kb DNA fragment included at the 5′ end in the vector, with the two fragments oriented in the same relative way as in the mouse genome; a gene encoding the diphtheria toxin A subunit (DTA) under transcriptional control of a modified herpes simplex virus type I thymidine kinase gene promoter coupled to two mutant transcriptional enhancers from the polyoma virus (523).

Mouse embryonic stem (ES) cells derived from C57B1/6NTac mice are transfected by electroporation with the 3′ vector (505) according to known procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the 3′ vector into their DNA by using the neomycin analogue drug G418. There is also negative selection for cells that have integrated the vector into their DNA but not by homologous recombination. Non-homologous recombination will result in retention of the DTA gene, which will kill the cells when the gene is expressed, whereas the DTA gene is deleted by homologous recombination since it lies outside of the region of vector homology with the mouse Igκ locus. Colonies of drug-resistant ES cells are physically extracted from their plates after they became visible to the naked eye about a week later. These picked colonies are disaggregated, re-plated in micro-well plates, and cultured for several days. Thereafter, each of the clones of cells is divided such that some of the cells could be frozen as an archive, and the rest used for isolation of DNA for analytical purposes.

DNA from the ES cell clones is screened by PCR using a gene-targeting assay. For this assay, one of the PCR oligonucleotide primer sequences maps outside the region of identity shared between the 3′ vector (505) and the genomic DNA (501), while the other maps within the novel DNA between the two arms of genomic identity in the vector, e.g., in the HPRT (545) or neomycin resistance (547) genes. These assays detect pieces of DNA that are only present in clones of ES cells derived from transfected cells that had undergone homologous recombination between the 3′ vector (505) and the endogenous mouse Igκ locus. PCR-positive clones are selected for expansion followed by further analysis using Southern blot assays.

The Southern blot assays are performed according to known procedures; they involve three probes and genomic DNA digested with multiple restriction enzymes chosen so that the combination of probes and digests allowed for conclusions to be drawn about the structure of the targeted locus in the clones and whether it is properly modified by homologous recombination. One of the probes maps to DNA sequence flanking the 5′ side of the region of identity shared between the 3′ kappa targeting vector (505) and the genomic DNA; a second probe also maps outside the region of identity but on the 3′ side; the third probe maps within the novel DNA between the two arms of genomic identity in the vector, e.g., in the HPRT (545) or neomycin resistance (547) genes. The Southern blot identifies the presence of the expected restriction enzyme-generated fragment of DNA corresponding to the correctly mutated, i.e., by homologous recombination with the 3′ kappa targeting vector (505) part of the kappa locus, as detected by one of the external probes and by the neomycin resistance or HPRT gene probe. The external probe detects the mutant fragment and also a wild-type fragment from the non-mutant copy of the immunoglobulin kappa locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Acceptable clones are then modified with the 5′ vector (503) using procedures and screening assays that are essentially identical in design to those used with the 3′ vector (505), except that puromycin selection is used instead of G418/neomycin selection, and the protocols are tailored to match the genomic region modified by the 5′ vector (503). The goal of the 5′ vector (503) transfection experiments is to isolate clones of ES cells that have been mutated in the expected fashion by both the 3′ vector (505) and the 5′ vector (503), i.e., doubly targeted cells carrying both engineered mutations. In these clones, the Cre recombinase causes a recombination (502) to occur between the loxP sites introduced into the kappa locus by the two vectors, resulting in the genomic DNA configuration shown at 507.

Further, the clones must have undergone gene targeting on the same chromosome, as opposed to homologous chromosomes; i.e., the engineered mutations created by the targeting vectors must be in cis on the same DNA strand rather than in trans on separate homologous DNA strands. Clones with the cis arrangement are distinguished from those with the trans arrangement by analytical procedures such as fluorescence in situ hybridization of metaphase spreads using probes that hybridize to the novel DNA present in the two gene targeting vectors (503 and 505) between their arms of genomic identity. The two types of clones can also be distinguished from one another by transfecting them with a vector expressing the Cre recombinase, which deletes the pu-Tk (539), HPRT (545) and neomycin resistance (547) genes if the targeting vectors have been integrated in cis, and comparing the number of colonies that survive ganciclovir selection against the thymidine kinase gene introduced by the 5′ vector (503) and by analyzing the drug resistance phenotype of the surviving clones by a “sibling selection” screening procedure in which some of the cells from the clone are tested for resistance to puromycin or G418/neomycin. Cells with the cis arrangement of mutations are expected to yield approximately 10³ more ganciclovir-resistant clones than cells with the trans arrangement. The majority of the resulting cis-derived ganciclovir-resistant clones should also be sensitive to both puromycin and G418/neomycin, in contrast to the trans-derived ganciclovir-resistant clones, which should retain resistance to both drugs. Clones of cells with the cis-arrangement of engineered mutations in the kappa chain locus are selected for further use.

The doubly targeted clones of cells are transiently transfected with a vector expressing the Cre recombinase (502) and the transfected cells are subsequently placed under ganciclovir selection, as in the analytical experiment summarized above. Ganciclovir-resistant clones of cells are isolated and analyzed by PCR and Southern blot for the presence of the expected deletion (507) between the two engineered mutations created by the 5′ vector (503) and the 3′ vector (505). In these clones, the Cre recombinase causes a recombination to occur between the loxP sites (537) introduced into the kappa chain locus by the two vectors. Because the loxP sites are arranged in the same relative orientations in the two vectors, recombination results in excision of a circle of DNA that includes the entire genomic interval between the two loxP sites. The circle does not contain an origin of replication and thus is not replicated during mitosis and is therefore lost from the clones of cells as they undergo clonal expansion. The resulting clones carry a deletion of the DNA that was originally between the two loxP sites. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

The ES cell clones carrying the deletion of sequence in one of the two homologous copies of their immunoglobulin kappa chain locus are retransfected (504) with a Cre recombinase expression vector and a vector (509) that includes a partly feline immunoglobulin kappa chain locus containing Vκ (551) and Jκ (555) gene segments. The key features of the vector are the following: a lox5171 site (531); a neomycin resistance gene open reading frame (547), lacking the initiator methionine codon, but in-frame and contiguous with an uninterrupted open reading frame in the lox5171 site (531) downstream of a methionine start codon (535); a FRT site (527); an array of 12 feline Vκ gene segments (551), each including feline coding sequences flanked on the 3′ side by mouse RSS and embedded in mouse noncoding sequences; optionally a 13.5 Kb piece of genomic DNA from immediately upstream of the cluster of J kappa region gene segments in the mouse kappa chain locus (not shown); DNA containing the five feline Jκ region gene segments (555) flanked on the 5′ side by mouse RSS and embedded in mouse noncoding DNA; a loxP site (537) in opposite relative orientation to the lox5171 site (531).

The sequences of the functional feline Vκ and Jκ gene coding regions are shown in SEQ ID NO. 14-30.

The transfected ES clones are placed under G418 selection, which enriches for clones of cells that have undergone RMCE, in which the donor DNA (509) that includes the partly feline immunoglobulin kappa chain locus is integrated in its entirety into the deleted endogenous immunoglobulin kappa chain locus between the lox5171 (531) and loxP (537) sites that were placed there by 5′ (503) and 3′ (505) vectors, respectively. Only cells that have properly undergone RMCE have the capability to express the neomycin resistance gene (547) because the promoter (529) as well as the initiator methionine codon (535) required for its expression are not present in the vector (509) and are already pre-existing in the modified host cell Igκ locus (507). The DNA region created using the 509 sequence is illustrated at 511. The remaining elements from the 5′ vector (503) located between the FRT sites (527) are removed via Flp-mediated recombination (506) in vitro or in vivo, as described below, resulting in the partly-feline immunoglobulin light chain locus as shown at 513.

G418-resistant ES cell clones are analyzed by PCR and Southern blotting to determine if they have undergone the expected RMCE process without unwanted rearrangements or deletions. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

The ES cell clones carrying the partly feline immunoglobulin kappa chain locus in the endogenous mouse immunoglobulin kappa chain locus (513) are microinjected into mouse blastocysts from strain DBA/2 to create partly ES cell-derived chimeric mice according to standard procedures. Male chimeric mice with the highest levels of ES cell-derived contribution to their coats are selected for mating to female mice. The female mice of choice for use in the mating are of the C57B1/6NTac strain, and also carry a transgene encoding the Flp recombinase that is expressed in their germline and will delete the FRT-flanked neomycin resistance gene (520) and other elements from the 5′ vector. Offspring from these matings are analyzed for the presence of the partly feline immunoglobulin kappa chain locus and for loss of the neomycin resistance gene. Mice that carry the partly feline immunoglobulin kappa chain locus are used to establish colonies of mice.

Mice carrying the partly feline immunoglobulin heavy chain locus, produced as described in Example 1, can be bred with mice carrying a partly feline immunoglobulin kappa chain locus. Their offspring are in turn bred together in a scheme that ultimately produces mice that are homozygous for both the partly feline Igh and the partly feline Igκ. Such mice produce partly feline heavy chains that include feline variable domains and mouse constant domains. They also produce partly feline kappa proteins that include feline kappa variable domains and the mouse kappa constant domain. Monoclonal antibodies recovered from these mice include feline heavy chain variable domains paired with feline kappa variable domains.

In one aspect, the mice that are homozygous for both the partly feline Igh and partly feline Igκ, are bred to mice homozygous for the partly feline lambda loci created in Example 3 to generate mice homozygous for all three loci.

Those skilled in the art will recognize that the 5′ vector (503) and subsequent strategy used here to target the Igκ locus can also be used in place of the 5′ vector (201) in FIG. 2 as an alternate strategy to target the Igh locus. In this case, the 5′ vector (503) is modified to replace the genomic DNA regions (525 and 541) homologous to the Igκ locus with genomic DNA regions (213 and 215 in FIG. 2) homologous to the Igh locus

### Example 3: Introduction of a Heterologous Partly Feline Immunoglobulin Locus into the Immunoglobulin Lambda Chain Gene Locus of a Mouse Genome

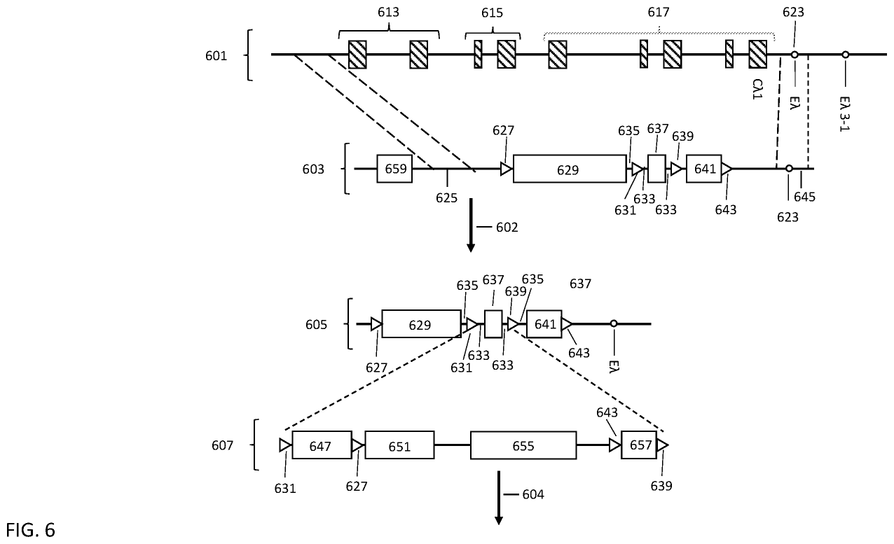

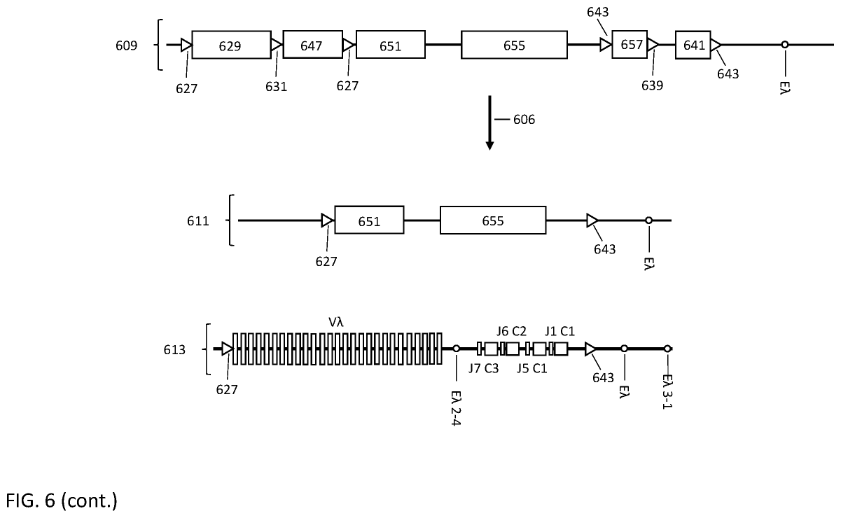

A method for replacing a portion of a mouse Igλ locus with partly feline Igλ locus is illustrated in FIG. 6. This method includes deleting approximately ˜200 Kb of DNA from the wild-type mouse immunoglobulin lambda locus (601 and FIG. 1, bottom) that includes Vλ2/Vλ3 gene segments (613), Jλ2/Cλ2 gene cluster (615), and Vλ1-Jλ3/Cλ3-Jλ1/Cλ1 gene cluster (617) by a homologous recombination process involving a targeting vector (603) that shares identity with the endogenous mouse immunoglobulin lambda locus both upstream of the Vλ2/Vλ3 gene segments (613) and downstream of the Cλ1 gene segment (rightmost box in 617) and either upstream or downstream of the Eλ enhancer (623). The vector replaces the ˜200 Kb of the endogenous mouse genomic DNA with elements designed to permit a subsequent site-specific recombination in which a heterologous immunoglobulin lambda locus replaces the modified Vλ locus via RMCE (604). In this example, the heterologous immunoglobulin lambda locus is a synthetic nucleic acid that includes feline Igλ coding sequences and mouse Igλ non-coding sequences.

The key features of the gene targeting vector (603) for accomplishing the ˜200 Kb deletion and inserting the site-specific recombination sites are as follows: a negative selection gene such as a gene encoding the A subunit of the diphtheria toxin (DTA, 659) or a herpes simplex virus thymidine kinase gene (not shown); 4 Kb of genomic DNA from 5′ of the mouse Vλ2/Vλ3 variable region gene segments in the immunoglobulin lambda locus (625); a FRT site (627); genomic DNA containing the mouse Polr2a gene promoter (629); a translation initiation sequence (methionine codon embedded in a “Kozak” consensus sequence) (635); a mutated loxP recognition sequence (lox5171) for the Cre recombinase (631); a transcription termination/polyadenylation sequence (633); an open reading frame encoding a protein that confers resistance to puromycin (637), whereas this open reading frame is on the antisense strand relative to the Polr2a promoter and the translation initiation sequence next to it and is followed by its own transcription termination/polyadenylation sequence (633); a loxP recognition sequence for the Cre recombinase (639); a translation initiation sequence (a methionine codon embedded in a “Kozak” consensus sequence) (635) on the same, antisense strand as the puromycin resistance gene open reading frame; a chicken beta actin promoter and cytomegalovirus early enhancer element (641) oriented such that it directs transcription of the puromycin resistance open reading frame, with translation initiating at the initiation codon downstream of the loxP site (635) and continuing back through the loxP site into the puromycin open reading frame all on the antisense strand relative to the Polr2a promoter and the translation initiation sequence next to it; a mutated recognition site for the Flp recombinase (643); genomic DNA (645) containing the Eλ enhancer element (623).

Mouse embryonic stem (ES) cells derived from C57B1/6NTac mice are transfected (602) by electroporation with the targeting vector (603) according to known procedures. Homologous recombination replaces the endogenous mouse immunoglobulin lambda locus with the site-specific recombination sites from the targeting vector (603) in the ˜200 Kb region resulting in the genomic DNA configuration depicted at 605.

Prior to electroporation, the vector DNA is linearized with a rare-cutting restriction enzyme that cuts only in the prokaryotic plasmid sequence or the polylinker associated with it. The transfected cells are plated and after ˜24 hours placed under positive drug selection using puromycin. There is also negative selection for cells that have integrated the vector into their DNA but not by homologous recombination. Non-homologous recombination will result in retention of the DTA gene (659), which will kill the cells when the gene is expressed, whereas the DTA gene is deleted by homologous recombination since it lies outside of the region of vector homology with the mouse Igλ locus. Colonies of drug-resistant ES cells are physically extracted from their plates after they became visible to the naked eye over a week later. These picked colonies are disaggregated, re-plated at limiting dilution in micro-well plates and cultured for several days. Thereafter, each of the clones of cells are divided such that some of the cells are frozen as an archive, and the rest used for isolation of DNA for analytical purposes.

DNA from the ES cell clones is screened by PCR using a known gene-targeting assay. For these assays, one of the PCR oligonucleotide primer sequences maps outside the regions of identity shared between the targeting vector and the genomic DNA, while the other maps within the novel DNA between the two arms of genomic identity in the vector, e.g., in the puro gene (637). These assays detect pieces of DNA that would only be present in clones of cells derived from transfected cells that had undergone homologous recombination between the targeting vector (603) and the endogenous DNA (601).

PCR-positive clones from the transfection are selected for expansion followed by further analysis using Southern blot assays. The Southern blots involve three probes and genomic DNA from the clones that has been digested with multiple restriction enzymes chosen so that the combination of probes and digests allow identification of whether the ES cell DNA has been properly modified by homologous recombination.

Karyotypes of the PCR- and Southern blot-positive clones of ES cells are analyzed using an in situ fluorescence hybridization procedure designed to distinguish the most commonly arising chromosomal aberrations that arise in mouse ES cells. Clones that show evidence of aberrations are excluded from further use. Karyotypically normal clones that are judged to have the expected correct genomic structure based on the Southern blot data are selected for further use.

The ES cell clones carrying the deletion in one of the two homologous copies of their immunoglobulin lambda chain locus are retransfected (604) with a Cre recombinase expression vector together with a vector (607) that includes a partly feline immunoglobulin lambda chain locus containing feline Vλ and Jλ region gene segment coding sequences. The key features of this vector (607) are as follows: a lox5171 site (631); a neomycin resistance gene open reading frame lacking the initiator methionine codon (647), but in-frame and contiguous with an uninterrupted open reading frame in the lox5171 site (631 in diagram 605)); a FRT site (627); an array of 32 functional feline lambda variable region gene segments, each gene segment including feline lambda coding sequences flanked on the 3′ side by mouse RSS and embedded in mouse lambda noncoding sequences (651); an array of J-C units where each unit includes a feline Jλ gene segment and a mouse lambda constant domain gene segment embedded within noncoding sequences from the mouse lambda locus (655), including the Eλ 2-4 enhancer element (FIG. 1). The feline Jλ gene segments are those encoding Jλ1, Jλ2 and Jλ4-11. The other Jλ gene segments, Jλ3 and Jλ12 are non-functional ORFs (Open Reading Frames), while the mouse lambda constant domain gene segments are Cλ1, Cλ2 or Cλ3 or a combination thereof; a mutated recognition site for the Flp recombinase (643); an open reading frame conferring hygromycin resistance (657), which is located on the antisense strand relative to the immunoglobulin gene segment coding information in the construct; a loxP site (639) in opposite relative orientation to the lox5171 site.

RCME inserts the partly feline immunoglobulin lambda chain locus from the RCME vector (607) into the modified endogenous mouse Igλ locus resulting in the genomic DNA configuration depicted at 609.

The sequences of the functional feline Vλ and Jλ gene coding regions are shown in SEQ ID NO. 31-73.

The transfected clones are placed under G418 or hygromycin selection, which enriches for clones of cells that have undergone a RMCE process, in which the partly feline immunoglobulin lambda chain variable is integrated into the deleted endogenous mouse immunoglobulin lambda chain locus between the lox5171 and loxP sites that were placed there by the gene targeting vector. The remaining elements from the targeting vector (603) are removed via FLP-mediated recombination (606) in vitro or in vivo (see below) resulting in the final partly feline immunoglobulin lambda chain locus as shown at 611.

A more detailed view of one configuration of the 611 partly feline immunoglobulin lambda chain locus is shown at 613 but is only provided as an example. Other arrangements and numbers of feline Vλ and Jλ gene segments and murine Cλ gene segments, as well as the position and number of enhancer elements are also possible.

G418/hygromycin-resistant ES cell clones are analyzed by PCR and Southern blotting to determine if they have undergone the expected recombinase-mediated cassette exchange process without unwanted rearrangements or deletions. Karyotypes of the PCR- and Southern blot-positive clones of ES cells are analyzed using an in situ fluorescence hybridization procedure designed to distinguish the most commonly arising chromosomal aberrations that arise in mouse ES cells. Clones that show evidence of aberrations are excluded from further use. Karyotypically normal clones that are judged to have the expected correct genomic structure based on the Southern blot data are selected for further use.

The ES cell clones carrying the partly feline immunoglobulin lambda chain locus (611) in the mouse immunoglobulin lambda chain locus are microinjected into mouse blastocysts from strain DBA/2 to create partially ES cell-derived chimeric mice according to known procedures. Male chimeric mice with the highest levels of ES cell-derived contribution to their coats are selected for mating to female mice. The female mice of choice here are of the C57B1/6NTac strain, which carry a transgene encoding the Flp recombinase expressed in their germline will delete the FRT-flanked selectable markers. Offspring from these matings are analyzed for the presence of the partly feline immunoglobulin lambda chain locus, and for loss of the FRT-flanked neomycin resistance gene and the mFRT-flanked hygromycin resistance gene that were created in the RMCE step. Mice that carry the partly feline immunoglobulin lambda chain locus are used to establish a colony of mice.

In one aspect, the mice homozygous for the partly feline immunoglobulin heavy chain locus and the partly feline immunoglobulin kappa light chain locus (as described in Examples 1 and 2) are bred to mice that carry the partly feline immunoglobulin lambda light chain locus. Mice generated from this type of breeding scheme are homozygous for the partly feline Igh locus and homozygous for the partly feline Igκ and Igλ loci. Monoclonal antibodies recovered from these mice include feline heavy chain variable domains paired in some cases with feline kappa variable domains and in other cases with feline lambda variable domains.

SEQUENCE INFORMATION
IGHV
SEQ ID NO 1: >LOC101082137
Atggagtttgtgctgggctgggttctcctggttgctcttttaaaaggtgtccagtgtga
cgtgcagctggtggagtctgggggagacctggtgaagcctggggggtccctgagactca
cctgtgtagcctctggattcaccttcagtagttccagcatgaactgggtccgccaggct
ccagggaaggggctgcagtgggtcgcatatatttatgatgatggaagtagcacatacta
cgcagactccgtgaagggccgattcaccatctccagagacaacgccaagaacacgctgt
atctgcagatgaacaacctgaagaccgaggacacggccacatattactgtgcaagagac
acggtgaggggacctcactgggagcccagacacaaacctccctgcaggaggggatcagc
accaccagggggcgcacactatgcacaactctggtttgtgttcccaggagcaggtgcag
atggaggttacaggcaggtttcctgtcagggtctggggcttcctctccacacagcagtt
tccccagggagcctctctggacacaggattctgtactttcctgttcattccttgactta
SEQ ID NO 2: >LOC101093781
Atggagtttgtgctgggctgggttttcctggttgctcttttaaaaggtgtccagtgtga
cgtgcagctggtggagtctgggggagacctggtgaagcctggggggtccctgagactca
cctgtgtggcctctggattcaccttcagtagctatggaatgagctgggtccgccaggct
ccagggaaggggctgcagtgggtcgcatatattagatatgatggaagtagcacatacta
cgcagactccgtgaagggccgattcaccatctccagagacaacgccaagaacacgctgt
atctgcagatgaacagcctgaagaccgaggacacggccacatattactgtgcaagagac
acgctgaggggacctcactgggagcccacacacaaacctccctgcaggaggggatcagc
accaccagggggcgcacactatgcacaattctggtttgtgttcccaggagcaggtgcag
atggaggttacaggcaggtttcctgtcagggtctggggcttcctctccacacagcagtt
tctccagggagcctctctggtcacaggattctgtgtttatctattaactctctgaatta
g
SEQ ID NO 3: >LOC111559009
atggagtttgtgctgggctgggttttcctggttgctcttttaaaaggtgtccagtgtga
cgtgcagctggtggagtctgggggagacctggtgaagcctggggggtccctgagactca
cctgtgtggcctctggattcaccttcagtagctactacatgaactgggtccgccaggct
ccagggaaggggctgcagtgggtctcatggattaatactgatggaagtagcacaagcta
cgcagactccgtgaagggccgattcaccatctccagagacaacgccaagaacacgctgt
atctgcagatgaacagcctcaagaccgaggacacggccacatattactgtgcgagagac
acagtgaggggacctcactgggagcccagacacaaacctccctgcaggaggggatcagc
accaccagggggcgcacactatgcacaattctcctttgtgttcccaggcgcaggtgcag
atggaggttacaggcaggtttcctgtcagggtctggggcttcctctccacacagcagtt
tccccagggagcctccctggttatatgattctgtgtttacctattaactctctgaatta
g
SEQ ID NO 4: >LOC111560938
atgcagatgctgtggtccctcctctgcctgctggcagctcccctgggtgtcctatctca
acttacacttcgggagtccggcccaggactggtgaagccttcacaatccctctctctca
cctgcgttgtctccggaggctctgttaccagcagttactactggaactggatccgccag
cgccctgggagagggttggagtggctggggtactggtcaggtagcaccagctacaaccc
ggctttccagggccgcatctccatcactgctgacacagcccagaaccagttctccctgc
agctgagctccatgaccaccgaggacacggccgtgtattactgtgcaagaagcacagtg
agggaaagtcagtgtgagctcagtcacaaaccttggtgcagggacctggaggggctggg
ctgcaggggcgctcaggatccacaagagggcacacaggacctaccaggggaactagggc
atcagggggtgcttagggccccttaccacagggaccagcccagaaacaggggcagagca
ggagtgaggtccccactgtcagtatctggagctttctcttcctggcactctgatcctat
ggggacctccctttctttcttgcttgcgttcccttttgtttcagtcccagtgtg
IGHD
SEQ ID NO 5: >DH206
GCATAGCGGAAGCTGGTCC
SEQ ID NO 6: >DH447
GGTAGTAGCGGGTGGGCT
SEQ ID NO 7: >DH1151
TTACTACGATAGCGACTATGCC
SEQ ID NO 8: >DH2663
TCTATAACTACGGGTGGTAC
IGHJ
SEQ ID NO 9: JH1
Cctatgattacttccagttttggggccagggcaccctggtcaccgtctcctcag
SEQ ID NO 10: JH2
caatacttttggtatctggggccaaggtacccaggtcaccgtctcccaag
SEQ ID NO 11: JH3
actactttgactactggggccaaggagccctggtgacggtgtcctcag
SEQ ID NO 12: JH4
Actactttgactactggggccaaggagccctggtgacggtgtcctcag
SEQ ID NO 13: JH5
attactacggtatcgatctctggggccatggaaccatagtcacagtgtcctcag
IGKV
SEQ ID NO 14: >IMGT000050|IGKV1-10*01
atgaaggcccccgctcagctcctgggcctcctgctgctctggctcccaggagccagctgc
gaaatccagatgacccagtctccatcctcgctgtctgcatctccaggagacagagtcacc
atcacctgccgggcgagtcagaacgttaacacgtggttagcctggtatcagcagaaaccg
gggaaagttcctaagcttctgatctatcgtgcatccacgttgcaaactggggtcccctcg
cggttcagcggcagtgggtctgggacagatttcaccctcaccatcagcagcctggagcct
gaagacgctgccacttactactgtcagcagcataacagcggcatc
SEQ ID NO 15: >IMGT000050|IGKV1-17*01
atgaaggcccccgctcagctcctgggcctcctgctgctctggctcccaggagccagctgt
gaaatccagatgacccagtctccatcctcgctgtctgcatctccaggagacagagtcacc
atcacctgccgggcgagtcagaatgttaacacgtggttagcctggtatcagcagaaaccg
gggaaagttcctaagcttctgatctatcgtgcatccacgttgcaaactggggtcccctcg
cggttcagcggcagtgggtctgggacagatttcaccctcaccatcagcagcctggagcct
gaagacgctgccacttactactgccagcaaagtagcaatctccctcc
SEQ ID NO 16: >IMGT000050|IGKV2-4*01
atgaggttccctgctcagctgctggggctgctgatgctctggatcccaggatccagtggg
gatgtcgtgatgacgcagacccctctgtccctgcccgtcacccctggagagccggcctca
atctcctgcagggccagtcagagcctcctgcacagtaatggaaatacttatctgaattgg
tacctgcagaagccaggccagtctccacggcgactgatctataaggtttccaaccgggac
tctggggtcccagacaggttcagtggcagcgggtcagggacagatttcaccctgagaatc
agcagggtggaggctgacgacgtcggagtttattactgccagcaaggtacacatgctcct
cg
SEQ ID NO 17: >IMGT000050|IGKV2-5*01
atgaggttccctgctcagctgctggggctgctgatgctctggatcccaggatccagtggg
gatgtcgtgatgacgcagacccctctgtccctgcccgtcacccctggagagccggcctca
atctcctgcagggccagtcagagcctcctgcacagtaatggaaatacttatctgaattgg
tacctgcagaagccaggccagtctccacggcgactgatctataaggtttccaaccgggac
tctggggtcccagacaggttcagtggcagcgggtcagggacagatttcaccctgagaatc
agcagggtggaggctgacgacgtcggagtttattactgccagcaaggtacacatgctcct
cg
SEQ ID NO 18: >IMGT000050|IGKV2-9*01
atgaggttccctgctcagctgctggggctgctgatgctctggatcccaggatccagtggg
gatgtcgtgatgacgcagacccctctgtccctgcccgtcacccctggagagccggcctca
atctcctgcagggccagtcagagcctcctgcacagtaatggaaacacctatttacattgg
tacctgcagaagccaggccagtctccacggcgactgatctatagggtttccaaccgggac
tctggggtcccagacaggttcagtggcagcgggtcagggacagatttcaccctgagaatc
agcagggggaggctgacgacgtcggagtttattactgcctgcaaggtacacacagacct
cc
SEQ ID NO 19: >IMGT000050|IGKV2-12*01
atgaggttccctgctcagctcctgggactcatcatgctctggatcccaggatccagtggg
gatattgtgatgacgcagacccctctgtccctgtccgtcacccctggagagccagcctca
atctcctgcagggccagtcagagcctcctgcacagtgatggaaatacttatctgaattgg
tacctgcagaagccaggccagtctccacggcgcttgatctatcttgtttccaaccgggac
tctggggtcccagacaggttcagtggcagcgggtcagggacagatttcaccctgagaatc
agcagggtggaggctgacgacgtcggtgtttattactgcggtcaagctttacagtatcct
CC
SEQ ID NO 20: >IMGT000050|IGKV2-13*01
atgaggttccctgctcagctcctggggctgctagtgctttggttccctggatccggtgcg
gatgtcgtgatgacacagacccctctgtccctgcctgtcacccctggagagccggcctca
atctcctgcagggccagtcagagcctcctgcacagtgatggaaatacttatctgaattgg
tacctgcagaagccaggccagtctccacggcgactgatctataaggtttccaaccgggac
tctggggtcccagacaggttcagtggcagcgggtcagggacagatttcaccctgagaatc
agcagggtagaggctgacgacgtcggagtttattactgcctgcaaggtacaaagtatcct
ac
SEQ ID NO 21: >IMGT000050|IGKV2-14*01
atgaggttccctgctcagctcctggggctgctagtgctttggttccctggatccagtggg
gatgtcatgatgacccagacccttctgtccctgcccgtcgcccctggacagccgggctca
atctcctgcagggccagtcagagcctcctgcccagcaatggatactcctatttaagttgg
tacctgcagaagacaggccagtctccacagcgcccgatctatcaggtttccaaccgggcc
tctggggtcccagacaggttcagtggcagcgggtcagggacagatttcacactcaaaatc
aacagagtggaggctgaggatgtgggagtttattgctgcttgcaagatatacaacttcct
ct
SEQ ID NO 22: >IMGT000050|IGKV4-1*01
atggtgacccagaggcaggtcctcctatccttgttgctgtgggtctcaggtgcctgtggg
gcgatcacgatgacgcagtctccaggctccctggctgggtctccaggtcagcaggtcacc
atgaactgcagggccagtcagagtgttagcagctacttagcctggtaccagcagaaacca
gggcagcatcctaagctgctcatctactcagcttccacccgggcatctggagtccccgac
cgattcagtggcagtgggtccgggacggatttcaccctcaccatcagcaacctccaggct
gaagacgtggcgagttactactgtcagcagtattacagctctcctcc
SEQ ID NO 23: >IMGT000050|IGKV6-6*01
atggtgtctccatcacagcttcttgggcttctgctcctctgggttccagcctccagtggt
gaggttgtgctgacccagtcctcagccttcctgtccaggactctaaaagaaaaagccacc
atcacctgccgagccaatcagggcatcagcaccatcttgcactggtatcagcagaaacca
aatcaggctccgaagctccttgtgaagtatgcttcccagtccgtctcgggagcgccgtcg
cggttcagcggcagtgggtctgggacagatttcaccctcaccatcagcagcccggagcct
gaagacgctgccacttactactgccagcaaagtaacaatctccctcg
SEQ ID NO 24: >IMGT000050|IGKV8-3*01
atgggggtcctgacccagctcctctgccttctgctggcctgcctcccagctgccggcggg
accactgagttgacccagtctcccacccatctctctgtgtccctgacagacagcgtgtcc
gtcatctgcagggccagtgagagcattagtgatcacttaagctggtatcagcagaaacca
ggccagcctcccaagcttatcatctatgatgccgataacctagagtctggcgtctcagac
cgcttctctgggattcagtctggcacagaattcatcctcaaaatcagcacagtcgaggct
gatgacgccgccgcttattactgccagcagggttatgcgcttcctcc
SEQ ID NO 25: >IMGT000050|IGKV8-16*01
atgggggtcctgacccagctcctctgccttctgctggcctgcctcccagctgccagcggg
accgctgagttgacccagtctcccacccatctctctgtgtccctgacagacagcgtgtcc
ctcatctgcagggccaatgagagcgttagtgattacttaagctggtatcagcagaaacca
ggccagcctcccaagcttatcatctatgatgccgataatctagagtctggcgtctcagac
cgcttctctgggattcagtctgacacagaattcatcctcaaaatcagcacagtcgaggct
gatgacgccgccatttattactgccagcaggattatgcgcttcctcc
IGKJ
SEQ ID NO 26: >IMGT000050|IGKJ1*01
gtggacgtttggccaaggaaccaagctggaagtcaaac
SEQ ID NO 27: >IMGT000050|IGKJ2*01
tgtacaatttcggccaggggacgaagctggagataaaac
SEQ ID NO 28: >IMGT000050|IGKJ3*01
gttcactttcggccaggggaccaaactggagatgaaac
SEQ ID NO 29: >IMGT000050|IGKJ4*01
gctcactttcggcccaggtaccaagctggagatcaaac
SEQ ID NO 30: >IMGT000050|IGKJ5*01
gaccacctttggccaagggacacatctggagattaaac
IGLV
SEQ ID NO 31: >IMGT000038|IGLV1-32*01
atggcttggtctccggtccttctcaccctcctcgctcactgcacagggtcctgggcccagtctgt
actggctcagccatcctcggtgtcaggctccttgggccagagggtcaccatctcctgctctggaa
gcagttccaacatcggtagcaattatgtgagctggtaccaacaactcccaggcacaacccccaaa
accataatctattgggataatagcagaccctcgggggtctctgaacgattctctggctccaagtc
tggcagcacaggcaccctgaccatcactgggctccaggctgaggacgaggctgattattactgct
cagcatgggatggtagtctgagagctca
SEQ ID NO 32: >IMGT000038|IGLV1-36*01
atggcctggtctcctctcctcctcatcctcctcgctcactgcacagtgtcctgggcccag
tctaggctgactcagccgccctcagtgtctggttctctgggccagagggtcaccatctcc
tgcgctggaagcagctctaatattggtggttatggtgtgaactggcaccaacaattccca
ggaatggcccccaaaaccatcatctatggtaatagcaatcgaccctctggggtcccagat
cgattctccggctccaagtctggcaacacaggcaccctgaccatcactgggctccaggct
gaggacgaggctgattattactgctcatcgtgggacagcagtagcagtgctcg
SEQ ID NO 33: >IMGT000038|IGLV1-36*01
atggcctggtctcctctcctcctcatcctcctcgctcactgcacagtgtcctgggcccag
tctaggctgactcagccgccctcagtgtctggttctctgggccagagggtcaccatctcc
tgcgctggaagcagctctaatattggtggttatggtgtgaactggcaccaacaattccca
ggaatggcccccaaaaccatcatctatggtaatagcaatcgaccctctggggtcccagat
cgattctccggctccaagtctggcaacacaggcaccctgaccatcactgggctccaggct
gaggacgaggctgattattactgctcatcgtgggacagcagtagcagtgctcg
SEQ ID NO 34: >IMGT000038|IGLV1-37*01
atggcctggtcccctctcctcctcaccctcctcattcactgcacagggtcctgggcccag
tctgtgttgagtcagccaccctcagtgtctggggccctgggccagacggtcaccatcccc
tgcgctggaggtgccaacaacatcggtattgctggtgggaactggtaccaacagcttcca
ggaaaggcccctaaactcctcatctatgatagtagcgatcgaccctcaggggtccctgaa
cgattctctggctccaagtctggcaacacaggctccttgaccatcactgggctccaggct
gaggacgaggctgattattactgccagtctcttgactttactcaaggtgctga
SEQ ID NO 35: >IMGT000038|IGLV1-40*01
atggcctggacccctcttctcctcaccctacttgctcactgtacagggtcctgggcccag
tctgtgctgactcagccaccatcagtgtctgggaccctaggccagaggatcaccatctcc
tgcaccggaagcagctccaacatcgggggtggtaatgctgtgagctggtaccaacaagtc
ccaggaatgggccccaaaaccgtcatctattggaataacagcaaaccctcgggggtccca
gatagattctccggctcaaagtctggcagttcaggcaccctgaccatcactgggctgcag
gctgaggacgaggctgattattactgctcagcgtgggatgatagtctcagtgctca
SEQ ID NO 36: >IMGT000038|IGLV1-42*01
atggcctggtctcctttccttctcactctcctcgctcactgcacagggtcctgggcccag
tctgtgctgactcagccgccctcagtgtcggggtccctgggccagagggtcaccatctcc
tgcactggaagcagctccaacatcgggggtggtaattatgtgagctggtaccaacaagtc
ccaggaacggcccccaggctcctgatttatgagaataacaaacgaccctccggggtcccc
gatcgattctctggctccaagtctggcagctcaggctccctgaccatcactgggctgcag
gctgacgacgaggctgattattattgtgcatcatgggacaatagtctcagtgctca
SEQ ID NO 37: >IMGT000038|IGLV1-51*01
atggcctggttccctcttctcctgacccttctcatccactgcacagggtcctgggcccag
tctgtgctgactcagccgccctcagtgtctggggccctggggcagacggtcaccatctcc
tgcgctggaagtaggagcaacatcggtattgctggtgtgaactggtaccaacagcttcca
ggaaaggcccctaaactcctctctatgctaatagacagaaacaaccctcatgggttccct
gaacgaatccgcgaatctcctggctccaagtctggcaacacaggctccttgaccatcact
gggctccaggctgaggacgaggctgattattactgctcagcatgggatgctattctgaaa
gctca
SEQ ID NO 38: >IMGT000038|IGLV1-56*01
atggcctggtcctttctcctcctcaccctcctcgctcactgcacagggtcctgggctcag
tctgtgctgactcagccgccctcagtgtctggggccctgggccagagggtcaccatctcc
tgcactggaagcagctccaacatcgggcgtggtaattatgtgagctggtaccaacaactc
tcaggaacagctcccaaactcctcatctatggtaatagcaatcgaccctcgggggtccca
gatcgattttctggctccaagtctggcagcacaggctccttgaccatcactgggctgcag
gctgaggacgaggctgattattactgtgcagcgtgggacagcagtctcaatgctca
SEQ ID NO 39: >IMGT000038|IGLV1-61*01
atgtcctggtctcctgtccttctcgccctcttcactcactgcacagggtcctgggcccag
tctgtactgactcagccaccctcggtgtcaggctccttgggccagagggtcaccatctcc
tgcactggaagcagctcccacatcagtaacaattttgtgaactggtaccaacaactccca
ggcacaacccccaaaaccataatcctttgggatgatagcagaccctcgggggtctctgaa
cgattctctggctccaagtctggcagcacaggcaccctgaccatcactgggctccaggct
gaggacgaggctgattattactgctcagcatgggatgatagtctgagagctca
SEQ ID NO 40: >IMGT000038|IGLV1-63*01
atggcctggttccctcttctcctcaccctcctcatctactgcacagggtcctgggcccag
tctgagctgactcagccgccctcagtgtctgggggcctgggccagacggtcaccatctcc
tgcgctggaagtaggagcaacattggtattgctggtgtgaactggtaccaacagcatcca
ggaaaggcccctaaactcctcatctatggtagtagcaatcgaccctcaggggtccctgac
agattttctggctccaagtctggcaacacaggctccttgaccatcactgggctccaggcc
gaggacgaggctgattattattgcctgtctgttgacgttacgcgaggtgctga
SEQ ID NO 41: >IMGT000038|IGLV1-70*01
atggcctggttccctcttctcctcaccctcctcatctactgcacaggttcctgggcccag
tctgagctgactcaaccgccctcagtgtctgggggcctgggccagacggtcaccatctcc
tgcgctggaagtgccaacaacatcggtagaattggtgtgaactggtaccaacagtttcca
ggaaaagcccctaaactcctcatctccgcttatagcaatcaaccctcacgggtttctggt
cgattttctggctccacgtctggcaacacaggctccttgaccatcactgggctccaggcc
gaggacgaggctgattattactgcacgtctgctgaccctattcaaagtgctca
SEQ ID NO 42: >IMGT000038|IGLV2-24*01
atggcctgggctctggtcctcctcagtctcctcactcaggacacagggtcctgggcccag
tctgccctgaatcagcctccctcactgtccggggatctgggacgcacagtcaccatctcc
tgtgctggcagcagcaatgacattgggagatatagtgacgtctcctggtaccaacagctc
gaaggcacatcccccaaactcctgattcataatgtaaattcccggccttcagggatccct
gatcgcttctctggctccaagtctggcaacacggcctccttgaccatctctgggctccag
gctgaagatgaggctgattattactgttgctcatatgctagtagtaatactctc
SEQ ID NO 43: >IMGT000038|IGLV3-2*01
atggcctggacccctcttctcctcagtatcctggctcactgcacaggttccatggcctcc
tacgtgctgacccagcccccgtcggtgtcagtgaacctgggacagacagccagaatcacc
tgtgggggaaacaacattggaagtaaacatgcttactggtaccagcagaagccaggccag
gcccccatgctggtcatttactatagcagcaaccggccctcagggatccctgaccgattc
tctggcaccaactcggggaacacggccaccctgaccatcagcggggcccaggctgaggac
gaggctgactattactgtcaggtgtgggataacagtggtaatgct
SEQ ID NO 44: >IMGT000038|IGLV3-5*01
atggcctggacccctctcctcctcagtgtcctggcatactacacaggctccgtgacctca
aacagggtgactcagcccccttccatttcagtggccctgggagagatggcaaggatcacc
tgtgagggaaacaacatcggaaatacatatgtttcctggtaccagcagaagccgaaccag
gtgcccctgatgattatttatcaggatagcaaccggccttcagggatccctgaccgattc
tctggctctaactctgggaacacggccaccctgactgtcagcggggcccgggctgaggat
gaggctgactattactgtctgtctgctcacagcagtagtaacgtt
SEQ ID NO 45: >IMGT000038|IGLV3-6*01
atggcctggacccctctcctcctcggcctcctcgctcactgcacaggttctgtggcttcc
tatgagctgactcagcccccatcagtgtcagtgaacctgggacagacggccaggatcaca
tgtggaggaaacaacattggaaataaagatgcttactggtaccagcagaagtcaggccag
gcccccatgctgattatctatgaggacagcaaacggccctcagggatccctgaccgattc
tctggcaccaactcagggaacatggccaccttgaccatcagcggggcccgggccgaagat
gaggctgactattactgccaggtgtgggacagcagtagtgatgct
SEQ ID NO 46: >IMGT000038|IGLV3-7*01
atggcctggacccctctcatcctcagcctcctcacttactgcacaggttccatagcctcc
tatgtgctgactcagcccccctcagtgtcggtgagcctgggacagacggccaggaccacc
tgtggaggaaacaacattgaaagcaaaagtgttcactggtaccagcagaagtcaggccaa
acccctgtgctgattatctatagtgatagcaaccggccctcaggaatccccgaccgattc
tcaggcaccaactcggggaacacggccaccctgaccatcagcggggcccgggccgaggac
gaggctgactattactgtcaggtgtgggacagtagtagtgatgct
SEQ ID NO 47: >IMGT000038|IGLV3-11*01
atggcctggacccctctcctgctccccctccttactctttgcacaggattcgtggcctcc
agtgaggtgactcagccgccctcagtgtcagtggccctgggacagacggctagaatcacc
tgctctggagatatgatggagaaaaaatataccaattggcaccagcagaagccaggtcaa
gcccccatacagatcatttataaggatagtgagcggccctcagggatccctgaccgattc
tctagctccagttcagggaaaacagtcaccctgaccatcagcggggcccgggccgaagac
gaggctgactactactgtcagtcttatgacatcagtagtaatgct
SEQ ID NO 48: >IMGT000038|IGLV3-13*01
atggcctggacccctctcctcctcggcctcctcgctcactgcacaggctccgtggcctcc
tatgtgctgactcagcccccatcagtggcagtgaacctgggacagacggccaggatcaca
tgtggaggaaacaacattggaagtagttatgcttactggtaccagcagaagtcaggccag
gcccctgtgctgattatctataaggatagcaaccggccctcagggatccctgaccgattc
tcaggcaccaactcggggaacacggccaccctgaccatcagcggggcccgggccgaggac
gaggctgactattactgtcagtcatatgacagcaactatgatcct
SEQ ID NO 49: >IMGT000038|IGLV3-17*01
atggcctggactcctctcctgctccccctccttattctctgcacaggttctgtgacagct
tctgaactgactcagccacctgcggtgtctgtggccttgggacagacggccacgattaca
tgcgagggagacagcttcgaaagcagtatgattaactggtatcagcagaaatcaggccaa
gcccccgtgctggtcatttatgaggatagtgagcagcccacagggattcctgacagattc
tctggctccaactcgggggatgcagccactctgaccatcaccggggcccaggctgaggac
gaggctgactattactgtcagtcctatgataacagtggtgatgct
SEQ ID NO 50: >IMGT000038|IGLV3-20*01
atggcctggacccctctcctccttggcctcctcgctcactgcacaggctccgtggcctcc
tatgtgctgactcagcccccatcagtggcagtgaacctgggacagacagccaggatcaca
tgtggaggaaacaacattggaagtagttatgcttactggtaccagcagaagtcaggccag
gcccctgtgctgattatctatgaggacagcaaacggccctcagggatccctgaccgattc
tcaggcaccaactcggggaacacggccaccctgaccatcagcggggcccgggccgaggac
gaggctgactattactgtcaggtgtgggataacagtggtaatgct
SEQ ID NO 51: >IMGT000038|IGLV3-23*01
atggcctggacccctctcctcctcggcctcctcgcttactgcacaggctccatggcctcc
tacgtgctgactcagcccccgtcagtgtcaatgaacctgggaaagacggccaggatcacg
tgtggaggaaacaacattggaagtaaatatgcttactggtaccagcagaagccaggccgg
gccccgatgatgattatctatgatgatagcaaaaggtcctcaaggacccctgaccgattc
tcaggcaccaactcggggaacacggccaccctgaccatcagcggggcccaggccgaggac
gaggccaactattactgtcaggtgtggggggcaatagtgatcct
SEQ ID NO 52: >IMGT000038|IGLV4-15*01
atggactgggctcccttctacctcctgcccttcattttctctacaggtttctgtgctctg
cctgtgctgacccaggctccatctgcatctgcctcacttggagcctcagtcaagctcacc
tgcaccctgagcagtgaacacagcaattactttgtttggtggtatcaacagagaccagag
aaggcccctcggtatttgatgaaggttaacagtgatggaagccacatcaaaggagacgga
atccccagtcgcttctcaggctcaagctctggggctgatcgctacttaaccatctccaac
atccagcctgaggatgaggctgactattactgtggtgagaagcatacaattgatggtcaa
accggttaagc
SEQ ID NO 53: >IMGT000038|IGLV5-30*01
atggcctggattcctcccctcctagtgctcctctgtcactgcacatgttccatctcccaa
tttgtggtgacccagccaccttccctctctgcatctctgggaacaacagccagactcacc
tgcaccctcaacagtgaaaggactgatatttaccccatattctggtaccagcaaaagcca
gggagccttcctcgttacctccttacctatgtaacagactcaaataagcatcaaggctct
ggggtccccagccgcttctctggatccaaagatacctctgccaatgcagggattttgctc
atttccgggattcaatcagaggatgaggcagactattactgtcagtcatttgatgttggt
gacgga
SEQ ID NO 54: >IMGT000038|IGLV5-34*01
atggcctggatccccatcctcctcgtgttcctctgtcactgcacaggttccctgtcccag
cctgtcgtgactcagccagcctccctctctgcatctctgggagccacagccagactcacc
tgcaccctgagcagggacatcaatgctggaggctactacatatactggtaccaacagaag
ccagggagccctccccggtatctcctgtactactactcagattcaaataagcaccagggc
cccggggtccccagccgcttctccgggtccaaagatgcctcggccaatgcagggcttctg
ctcatctctgggctgcagcctgaggacgaggctgactattactgtgcaacttggcacagt
agtgctggt
SEQ ID NO 55: >IMGT000038|IGLV5-46*01
atggcctggacccatgctctcctcgtgctcttctgccactgcacaggttccctgtcccag
cctgtcgtgactcagccaccctccctctctgcatctctgggagcaacagccagactcacc
tgcaccctcagcagggaagtgagcgttggtagtaaaagcatatactggtaccaacagaag
ccagggagccctcctcggtatttcctgtactactactcagactccagcaatgagctggga
cccggggtccccagtcgagtgtctgggtccaaagatgcctcggccaatgcagggcttctg
ctcatctctgggctgcagcctgaggacgaggctgactattactgtgctatagcacatggc
agtggaagcagctttggttact
SEQ ID NO 56: >IMGT000038|IGLV5-48*01
atggcttggactcttctagtcctcatgcttgtgtctcaatggacaggttccctgtcccag
cctgtgctgacccagccgtcctccctgtctgcatctcctggaacaacagccagactcacc
tgcaccctgagcagcgggttcagtgttggaggctactacataaattggttccagcagaag
ccagggagccctccccggtatctcctgtactactactcagattcaaataagcaccagggc
cctggggtccccagccgcttctccgggtccaaagatgcctcggccaatgcagggcttctg
ctcatctctgggccgcagcctgaggacgaggctgactattactgtgctatagcacatggc
agtggaagcagctaccgttact
SEQ ID NO 57: >IMGT000038|IGLV5-49*01
atggcctggatccccgtcctcctcgtgctcctctgtcactgcgcaggttccctgtcccag
cctgtcgtgactcagccaccctccctctctgcatctctgggagcaacagccagactcacc
tgcaccctcagcagggaagtgagcgttggtagtaaaagcatatactggtaccaacagaag
ccagggagccctcctcggtatttcctgtactactactcagactccagcaatgagctggga
cccggggtccccagtcgagtgtctgggtccaaagatgcctcggccaatgcagggcttctg
ctcatctctgggctgcagcctgaggacgaggctgactattactgtgctatagcacatggc
agtggaagcagctaccgttact
SEQ ID NO 58: >IMGT000038|IGLV5-55*01
atggcctggactcctgtcctcctcatgctcctctcccattgtgcaggttccctgtcccag
cctgtgctgacccagccgtcctccctgtctgcatctccgggaacaacagccagactcacc
tgcaccctgagcagcggcttcaatgttggaggctactacataagttggttccagcagaag
ccagggagccctccccggtatctcctgtactactactcagactcagataagcaccagggc
cccggggtccccagccgcttctccgggtccaaagatgcctcggccaatgcagggcttctg
ctcatctctgggccgcagcctgaggacgaggctgactattactgtgctacagatcatggc
agtggttact
SEQ ID NO 59: >IMGT000038|IGLV5-66*01
atggcttggcctcttctcgtcctcatactcctgtctcactgcacaggttccctgtcccag
cctgtgctgacccagccgccctccctgtctgcatctccgggaacaacagccagactcacc
tgcaccctgagcagcggcttcaatgttggaggctactacataagttggttccagcagaag
ccagggagccctccccggtatctcctgtactactactcagactcagataagcaccagggc
cccggggtccccagccgcttctccgggtccaaagatgcctcggccaatgcagggcttctg
ctcatctctgggccgcagcctgaggacgaggctgactattactgtgctacaggttatggc
agtgggagcagctaccgttact
SEQ ID NO 60: >IMGT000038|IGLV5-68*01
atggcctggatctccgtcctcctcgtgctcctctgtcactgcgcaggttccctgtctcag
cctgtcgtgactcagccagcctccctctctgcatctctgggagcaacagccagactcacc
tgcacgctgagcagggacatcaatgttggaggctactacatatactggtaccaacagaat
ccagggagccctccccggtatctcctgtactactactcagactccagtacacagttggga
cctggggtccccagccgcttctccggatccaaagatgcctcggccaatgcagggcttctg
ctcatctctgggctgcagcctgaggacgaggctgactattactgtgcaatcgggcacagt
agtgctggt
SEQ ID NO 61: >IMGT000038|IGLV5-71*01
atggcctggatccccatcctcctcgtgctcctctgtcactgcacaggttccctgtcccag
cctgtcttgactcagccagcctccctctctgcatctctgggagcaacagccagactcacc
tgcaccctgagcagggacatcaacgttggaagctataacatatactggtaccaacagaag
ccagggagccctccccggtatctcctgtactactactcagactcagataagcaccagggc
cctggcgtccccagccgcttctctgggtccaaagatgcctcggccaatgcagggcttctg
ctcatctctgggctgcagcctgaggacgaggctgactattactgtgcaatctggcacagt
agtgctggt
SEQ ID NO 62: >IMGT000038|IGLV5-79*01
atggcttggaccccttttttccttgtgttcctggctcactgcacaggttccctgtctcag
ccggtgctgacccagccaccctccctctctgcatctctgggaacttctgtgagacttacc
tgtaccctgagcagtggcttcagagttggtgatttctggataaactggtaccagcagaat
ccagggaaccctccccggtatctcctgtactaccactcagactcagataaacaccagggc
tccggggtccccagccgcttctctggatccagtgatgcctcggccaatgcagggcttctg
ctcatctctgggctgcagcctgaggatgaggctgactattactgtagcacatggcatggc
aactctaagtctta
SEQ ID NO 63: >IMGT000038|IGLV12-26*01
atggcctgggctcttctcctgttcacacttctgtctcactgcacaggggccacttcccag
gaagtagtgactcaggaaacttcactctcaacaactcctggaggaacagtcacactcacc
tgtggctccagtactggggctgtcaccaccagtaattatgccagctgggtccaacagaag
ccctaccagagattccagggtctgataggtgggaccagctaccggaacccaggggtccct
gcccgattctctggctccctggttggacagaaggccgtcctcaccatcacgggggcgcag
tcagaggatgaagctgagtattactgtgttctgtggttcagcaaccattac
IGLJ
SEQ ID NO 64: >IMGT000038|IGLJ1*01
ttgggtgttcggcggaggtacccatctgagcgtcctag
SEQ ID NO 65: >IMGT000038|IGLJ2*01
tcatattttcggtggagggacccatctgactgtcctcg
SEQ ID NO 66: >IMGT000038|IGLJ4*01
ttatgttttcggcggagggaccaaggtgaccgtcctcg
SEQ ID NO 67: >IMGT000038|IGLJ5*01
tcctattttcggcggagggacccgtctgaccgtcctcg
SEQ ID NO 68: >IMGT000038|IGLJ6*01
ttttgtttttggcagagggacctggctgacggtcctag
SEQ ID NO 69: >IMGT000038|IGLJ7*01
tgctttgttcggcggagggacccatctgaccgtcctcg
SEQ ID NO 70: >IMGT000038|IGLJ8*01
ttgggtgtttggcgatggaacccagctgactgtattag
SEQ ID NO 71: >IMGT000038|IGLJ9*01
tattgtgttcggcggagggacccatctgaccgtcctcg
SEQ ID NO 72: >IMGT000038|IGLJ10*01
ttgggtgtttggcgatggaacccagctgactgtattag
SEQ ID NO 73: >IMGT000038|IGLJ11*01
tattgtgttcggcggagggacccatctgaccgtcctcg

### Pre-D

This is a 21609 bp fragment upstream of the Ighd-5 D_{H} gene. The pre-D sequence can be found in Mus musculus strain C57BL/6J chromosome 12, Assembly: GRCm38.p4, Annotation release 106, Sequence ID: NC_000078.6

The entire sequence lies between the two 100 bp sequences shown below:

Upstream of the Ighd-5 D_{H} gene segment, corresponding to positions 113526905-113527004 in NC_000078.6:

SEQ ID NO 74:
ATTTCTGTACCTGATCTATGTCAATATCTGTACCATGGCTCTAGCAGAGA
TGAAATATGAGACAGTCTGATGTCATGTGGCCATGCCTGGTCCAGACTTG

2 kb upstream of the Adam6a gene corresponding to positions 113548415-113548514 in NC_000078.6:

SEQ ID NO 75:
GTCAATCAGCAGAAATCCATCATACATGAGACAAAGTTATAATCAAGAAA
TGTTGCCCATAGGAAACAGAGGATATCTCTAGCACTCAGAGACTGAGCAC

### Adam6a

Adam6a (a disintegrin and metallopeptidase domain 6A) is a gene involved in male fertility. The Adam6a sequence can be found in Mus musculus strain C57BL/6J chromosome 12, Assembly: GRCm38.p4, Annotation release 106, Sequence ID: NC_000078.6 at position 113543908-113546414.

Adam6a sequence ID: OTTMUSG00000051592 (VEGA)

1. A transgenic rodent with a genome in which an endogenous a rodent immunoglobulin variable gene locus is deleted and replaced with a partly feline immunoglobulin locus comprising feline immunoglobulin variable gene coding sequences and non-coding regulatory sequences based on the endogenous rodent immunoglobulin variable gene locus, wherein the partly feline immunoglobulin locus of the transgenic rodent is functional and expresses immunoglobulin chains comprising feline variable domains and rodent constant domains.

2. The transgenic rodent of claim 1, wherein the partly feline immunoglobulin locus comprises feline V_{H}, D_{H} and J_{H} coding sequences.

3. The transgenic rodent of claim 1, wherein the partly feline immunoglobulin locus comprises feline kappa V_{L} and J_{L} coding sequences, feline lambda V_{L} and J_{L} coding sequences, or a combination thereof.

4. The transgenic rodent of claim 1, wherein the partly feline immunoglobulin loci comprise feline V_{H}, D_{H} and J_{H}, feline kappa V_{L} and J_{L} coding sequences, feline lambda V_{L} and J_{L} coding sequences, or a combination thereof.

5. The transgenic rodent of any of claims 1 to 4, wherein the rodent is a mouse.

6. The transgenic rodent of any of claims 1 to 5, wherein the non-coding regulatory sequences comprise promoters preceding individual V gene segments, splice sites, and recombination signal sequences for V(D)J recombination.

7. The transgenic rodent of any of claims 1 to 6, wherein the partly feline immunoglobulin locus further comprises an ADAM6 gene.

8. The transgenic rodent of any of claims 1 to 7, wherein the partly feline immunoglobulin locus further comprises Pax-5-Activated Intergenic Repeat (PAIR) elements.

9. The transgenic rodent of any of claims 1 to 8, wherein the partly feline immunoglobulin locus further comprises CTCF binding sites from a heavy chain intergenic control region 1.

10. A cell of B lymphocyte lineage from the transgenic rodent of any of claims 1 to 9.

11. A hybridoma cell derived from the cell of B lymphocyte lineage of claim 10.

12. An immortalized cell derived from the cell of B lymphocyte lineage of claim 10.

13. A part of or whole immunoglobulin molecule comprising feline variable domains and rodent constant domains derived from the cell of B lymphocyte lineage of claim 10, the hybridoma of claim 11 or the immortalized cell of claim 12.

14. A method for generating the transgenic rodent of claim 1, the method comprising:
a) integrating in a rodent cell's genome at least one target site for a site-specific recombinase upstream of an endogenous immunoglobulin variable gene locus and at least one target site for a site-specific recombinase downstream of the endogenous immunoglobulin variable gene locus, wherein the endogenous immunoglobulin variable locus comprises (i) V_{H}, D_{H} and J_{H} gene segments, (ii) Vκ and Jκ gene segments, (iii) Vλ and Jλ gene segments; or (iv) Vλ and Jλ gene segments and Cλ genes;
b) providing a vector comprising a partly feline immunoglobulin locus, the partly feline immunoglobulin locus comprising partly feline immunoglobulin variable region gene segments, wherein each of the partly feline immunoglobulin variable region gene segments comprises feline immunoglobulin variable region gene coding sequences and rodent non-coding regulatory sequences, with the partly feline immunoglobulin variable region gene locus being flanked by target sites for a site-specific recombinase, wherein the target sites are capable of recombining with the target sites introduced into the rodent cell in step a);
c) introducing into the cell the vector of step b) and a site-specific recombinase capable of recognizing the target sites;
d) allowing a recombination event to occur between the genome of the cell and the partly feline immunoglobulin locus, resulting in a replacement of the endogenous immunoglobulin variable gene locus with the partly feline immunoglobulin locus;
e) selecting a cell that comprises the partly feline immunoglobulin variable locus generated in step d); and
f) utilizing the cell to create a transgenic rodent comprising the partly feline immunoglobulin variable locus.

15. The method of claim 14, wherein the cell is a rodent embryonic stem (ES) cell.

16. The method of claim 15, wherein the cell is a mouse embryonic stem (ES) cell.

17. The method of any of claims 14 to 16, further comprising, after the introducing step and before the providing step, a step of deleting the endogenous immunoglobulin variable gene locus by introduction of a recombinase that recognizes a first set of target sites, wherein the deleting step leaves in place at least two target sites in the rodent cell's genome that are not capable of recombining with one another.

18. The method of any of claims 14 to 17, wherein the vector comprises feline V_{H}, D_{H}, and J_{H}, coding sequences.

19. The method of any of claims 14 to 18, wherein the vector comprises V_{L} and J_{L} coding sequences, either κ or λ.

20. The method of any of claims 14 to 19, wherein the vector further comprises V gene promoters, splice sites, and recombination signal sequences of endogenous host origin.

21. The method of any of claims 14 to 20, wherein the vector further comprises an ADAM6 gene.

22. The method of any of claims 14 to 21, wherein the vector further comprises Pax-5-Activated Intergenic Repeat elements.

23. The method of any of claims 14 to 22, wherein the vector further comprises CTCF binding sites from a heavy chain intergenic control region 1.

24. A method of producing an antibody for therapeutic or diagnostic use, the method comprising:
(i) expressing an antibody with a feline variable domain cloned from an antibody-producing cell of a transgenic rodent whose genome comprises an endogenous rodent immunoglobulin locus variable region that has been deleted and replaced with an immunoglobulin locus variable region comprising at least one of each of a chimeric V_{H}, D and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises feline V, D or J immunoglobulin variable region coding sequences and rodent immunoglobulin variable region non-coding gene segment sequences; and
(ii) isolating the antibody with the feline variable domain, wherein the antibody is suitable for therapeutic or diagnostic use.

25. The method of claim 24, wherein the antibody is cloned from a B cell of the transgenic rodent.

26. A therapeutic or diagnostic antibody produced by the method of claim 24 or 25.

27. A method of producing a therapeutic or diagnostic antibody with feline variable domains, the method comprising:
(i) cloning a feline variable domain of an antibody expressed by an antibody-producing cell from a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises feline V, D or J immunoglobulin variable region coding sequences and rodent immunoglobulin variable region non-coding gene segment sequences; and
(ii) producing the therapeutic or diagnostic antibody comprising the feline variable domain of the antibody expressed by the transgenic rodent.

28. The method of claim 27, wherein the feline variable domain is cloned from an antibody expressed by a B cell from the transgenic rodent.

29. A therapeutic or diagnostic antibody produced by the method of claim 27 or 28.

30. A method of producing a monoclonal antibody comprising a feline variable domain, the method comprising:
(i) providing B cells from a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises feline V, D or J immunoglobulin variable region coding sequences embedded in rodent immunoglobulin variable region non-coding gene segment sequences;
(ii) immortalizing the B cells; and
(iii) isolating monoclonal antibodies comprising feline variable domains expressed by the immortalized B cells, or genes encoding the antibodies.

31. The method of claim 30, further comprising:
(iv) cloning the feline variable domains expressed by the B cells; and
(v) producing a therapeutic or diagnostic antibody comprising the feline variable domain cloned from the B cells of the transgenic rodent.

32. A method of producing antibodies comprising feline variable domains, the method comprising providing a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises feline V, D or J immunoglobulin variable region coding sequences embedded in rodent immunoglobulin variable region non-coding gene segment sequences, wherein the immunoglobulin locus of the transgenic rodent expresses antibodies comprising feline variable domains.

33. The method of claim 32, further comprising isolating the antibodies comprising feline variable regions expressed by the transgenic rodent, or genes encoding the antibodies.

34. The method of claim 32, further comprising:
(i) obtaining B cells from the transgenic rodent expressing antibodies specific for the target antigen;
(ii) immortalizing the B cells; and
(iii) isolating antibodies specific for the target antigen from the immortalized B cells.

35. The method of claim 34, further comprising cloning feline variable regions from the B cells specific for the particular antigen.

36. The method of claim 35, further comprising producing a therapeutic or diagnostic antibody using the feline variable regions cloned from the B cells.

37. A therapeutic or diagnostic antibody produced by the method of any of claims 32 to 36.
